(19) United States
(12) Patent Application Publication
(10) Pub. No.: US 2024/0101564 A1
(43) Pub. Date: Mar. 28, 2024

(54) SALTS OF A PI3Kdelta INHIBITOR, CRYSTALLINE FORMS, METHODS OF PREPARATION, AND USES THEREFORE

(71) Applicant: BeiGene Switzerland GmbH, Basel (CH)

(72) Inventors: Jing LI, Beijing (CN); Xiaosong YU, Beijing (CN); Xiaopeng CAI, Beijing (CN); Zhiwei WANG, Beijing (CN)

(73) Assignee: BeiGene Switzerland GmbH, Basel (CH)

(21) Appl. No.: 18/517,894

(22) Filed: Nov. 22, 2023

Related U.S. Application Data
(63) Continuation of application No. PCT/CN2022/095129, filed on May 26, 2022.

(30) Foreign Application Priority Data
May 27, 2021 (WO) PCT/CN2021/096509

(51) Int. Cl. C07D 487/04; C07C 53/128; C07C 57/15; C07C 59/255; C07C 65/05; C07D 213/80

(52) U.S. Cl. CPC C07D 487/04; C07C 53/128; C07C 57/15; C07C 59/255; C07C 65/05; C07D 213/80; C07B 2200/13

(57) ABSTRACT

The present invention relates to salts of a PI3Kdelta inhibitor (referred to as “Compound A” hereinafter), preferably fumarate, and the crystalline forms thereof. The present invention also relates to the process of preparation and uses of the salts and crystalline forms of Compound A.

### FIELD OF THE INVENTION

The present invention relates to salts of a PI3Kdelta inhibitor (referred to as “Compound A” hereinafter), preferably fumarate, and the crystalline forms thereof. The present invention also relates to the process of preparation and uses of the salts and crystalline forms of Compound A.

### BACKGROUND OF THE INVENTION

Phosphatidylinositol-4,5-bisphosphate 3-kinase δ (PI3Kδ) is frequently active in B-cell malignancies and is central to multiple signaling pathways that drive proliferation, survival, homing, and retention of malignant B-cells in lymphoid tissue and bone marrow. In B-cell malignancies, PI3K pathway activity is significantly elevated, which is driven by altered B-cell receptor (BCR) signaling together with other co-stimulatory signals present in lymphoid tissues such as chemokines and cytokines (Puri and Gold 2012, Okkenhaug and Vanhaesebroeck 2003). PI3Kδ functions to integrate and transduce these signals from the microenvironment, thus promoting malignant B-cell proliferation, growth, survival, adhesion, and homing, making it an attractive drug target for B-cell malignancies (Yang et al 2015).

PI3Kδ is also important for the homeostasis and function of T-regulatory (Treg) cells (Lim and Okkenhaug 2019). The inactivation of PI3Kδ in mice can stimulate immune responses against solid tumors via the inhibition of Treg cells (Ali et al 2014). With PI3Kδ expression at low or undetectable levels in most organs, inhibitors against PI3Kδ should be selective for the immune system and less toxic (Okkenhaug and Fruman 2010).

Because of the specific and critical functions of PI3Kδ in adaptive immune responses, inhibitors of PI3Kδ are being developed for the treatment of autoimmune and inflammatory disorders, hematologic and solid tumors, and activated PI3Kδ syndrome (Lucas et al 2016; Okkenhaug and Burger 2016). PI3Kδ inhibitors are also being developed for the treatment of solid tumors because PI3Kδ is essential for the homeostasis and function of Foxp3+ Treg cells (Patton et al 2006). Loss of PI3Kδ activity, especially by specific deletion in Treg cells, can restrict the growth of transplanted tumors in mice (Ali et al 2014), providing a rationale for the evaluation of PI3Kδ inhibitors in solid tumors.

WO2019/047915A1 disclose a series of PI3Kδ inhibitors, in particular (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide

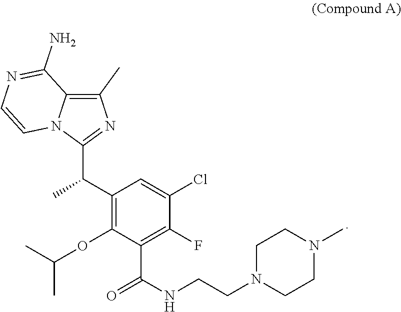 [SMILES: Cc1nc([C@@H](C)c2cc(Cl)c(F)c(C(=O)NCCN3CCN(C)CC3)c2OC(C)C)n2ccnc(N)c12]

Compound A is a potent and selective inhibitor of PI3Kδ in biochemical and cellular assays, it inhibits cellular growth of several cancer cell lines in vitro and induces dose-dependent antitumor effects against tumor xenografts engrated either subcutaneously or systemically in mice.

Compound A was confirmed to be amorphous (as shown in FIG. 49) and to have an aqueous solubility between 7.7 and 23.0 at room temperature. Compound A in the amorphous form has been found to be very viscous, which presents many challenges for the subsequent pharmaceutical formulation, transportation, storage, and administration, especially on the large scale.

In order to be manufactured into pharmaceutical products, it is strictly required that the active ingredient must have high purity and stability. Particularly, in order to maintain high stability in a longer shelf period, the active ingredient must have low hygroscopicity so that the influence on the quality by moisture can be avoided. Thus, the free base of Compound A needs to be converted into other forms such as salt to pursue improved properties.

For orally administered solid formulations comprising the desired active ingredient, the active ingredient needs to have the desired bioavailability so that the active ingredient could be absorbed into the blood circulation of the body as much as possible. However, the relationship between the bioavailability and the specific salt is unknown in the art, and a new salt of Compound A with higher bioavailability is highly desired.

Therefore, it remains the need for the discovery of new solid forms of Compound A or the salts thereof to meet the above pharmaceutical formulation requirements.

### SUMMARY OF THE INVENTION

The present application discloses an invention to address the foregoing challenges and needs by providing stable salts of Compound A, and especially fumarate of Compound A, which shows the desired crystallinity and improved bioavailability suitable for pharmaceutical formulation.

In addition, the inventors have found that among different salts of Compound A, fumarate salt of Compound A shows unpredictable high bioavailability, which makes the fumarate salt of Compound A suitable for pharmaceutical formulation.

Surprisingly, salts of Compound A, preferably fumarate salt of Compound A, even more preferably the crystalline of fumarate is a solid with very low viscosity. The salts of Compound A, preferably fumarate salt of Compound A, even more preferably the crystalline of fumarate can be used in the large-scale production of formulation process without the viscous problem.

Even more surprisingly, the fumarate salt type D showed an excellent long-term stability during the 3-month experiment. From the current data, we also could expect that fumarate salt type D should have a very good long-term stability, such as 6-month long-term stability, 12-month long-term stability, 24-month long-term stability and 36-month long-term stability.

Before the filing date of the instant application, the inventors of the instant application have unexpectedly found that only fumaric acid can form crystalline forms with the desired crystallinity, high stability, low hygroscopicity and low viscosity. with Compound A.
1. A pharmaceutically acceptable salt of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide, wherein said pharmaceutically acceptable salts are conventional inorganic salt(s) or organic salt(s).
2. The salt according to Item 1, which is in solid state.
3. The salt according to Item 1 or 2, wherein the salt is inorganic salt selected from hydrochloride, sulphate, phosphate, hydrobromide and/or nitrate; or is organic salt selected from fumarate, tartrate (L-tartrate or D-tartrate), laurate, stearate, gentisate, nicotinate, aspartate, succinate, adipate, malate (L-malate), citrate, glycolate, gluconate (D-gluconate), lactate (DL-lactate), acetate, benzene sulfonate, methanesulfonate, mesylate, benzoate, naphthalene sulfonate, and/or oxalate.
4. The salt according to Item 3, wherein the salt is selected from fumarate, L-tartrate, D-tartrate, sulphate, tartrate, laurate, stearate, gentisate, or nicotinate, preferably, is selected from fumarate or D-tartrate.
5. The salt according to Item 4, wherein the salt is fumarate.
6. The salt according to Item 5, wherein the salt is a compound of Formula (I):

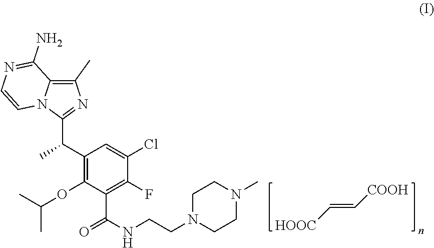

wherein n is a number from about 0.5 to about 2.0.
7. The salt according to Item 6, wherein n is a number about 0.5 to about 1.5; preferably n is a number selected from the group consisting of 0.5±0.1, 1.0±0.2 and 1.5±0.2.
8. The salt according to Item 7, n is a number selected from 1.0±0.1, 1.1±0.1 and 1.5±0.1; preferably, n is 0.95˜1.05, 1.05˜1.15 or 1.45˜1.55; more preferably, n is 0.98˜1.02, 1.08˜1.12 or 1.48˜1.52; even more preferably, n is 1.0, 1.1 or 1.5.
9. The salt according to Item 4, wherein the salt is tartrate, preferably the salt is D-tartrate.
10. The salt according to Item 9, wherein the salt is a compound of Formula (II):

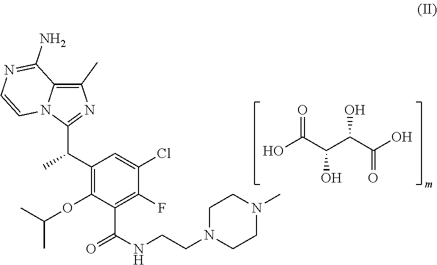

wherein m is a number from about 0.5 to about 2.0.
11. The salt according to Item 10, wherein m is a number about 0.5 to about 1.5; preferably m is a number selected from the group consisting of 0.5±0.1, 1.0±0.2 and 1.5±0.2.
12. The salt according to Item 10, m is a number selected from 1.0±0.1 and 1.5±0.1; preferably, m is 0.95˜1.05 or 1.45˜1.55; more preferably, m is 0.98˜1.02 or 1.48˜1.52; even more preferably, m is 1.0, or 1.5.
13. A pharmaceutical composition comprising a therapeutically effective amount of the salts according to any one of Items 1-12, and optionally one or more pharmaceutically acceptable carrier(s).
14. A method for treating or preventing a disorder or a disease selected from inflammatory disorder, autoimmune disease, or a cancer, comprising administering a subject in need thereof a therapeutically effective amount of the salts according to any one of Items 1-12, or the pharmaceutical composition of Item 13.
15. A process for the preparation of the salts of any one Items 1-12, comprising:
(a). Mixing the free base of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide and corresponding acid in an appropriate solvent to form a suspension;
(b). isolating the solid from the suspension to obtain the salt of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide.
16. The process according to Item 15, wherein the corresponding acid is selected from hydrochloric acid, sulfuric acid, phosphoric acid, hydrobromic acid, nitric acid, fumaric acid, L-tartaric acid, D-tartaric acid, lauric acid, stearic acid, gentistic acid, nicotinic acid, aspartic acid, succinic acid, adipic acid, malic acid (L-malic acid), citric acid, ascobic acid (L-ascobic acid), glycolic acid, gluconic acid (D-gluconic acid), lactic acid (DL-lactic acid), acetic acid, benzene sulfonic acid, methanesulfonic acid, benzoic acid, naphthalene sulfonic acid, and/or oxalic acid.
17. The process according to Item 16, wherein the corresponding acid is selected from sulfuric acid, fumaric acid, L-tartaric acid, D-tartaric acid, lauric acid, stearic acid, gentistic acid and/or nicotinic acid; preferably is fumaric acid.
18. The process according to any one of Items 15-17, wherein the selected from acetone, heptane (n-heptane), isopropyl alcohol, isopropyl acetate and/or 1,4-dioxane, and a combination thereof.
19. The process according to any one of Items 15-18, further comprising step (c) drying the solid in vacuum.
20. A crystalline form of a salt of Formula III

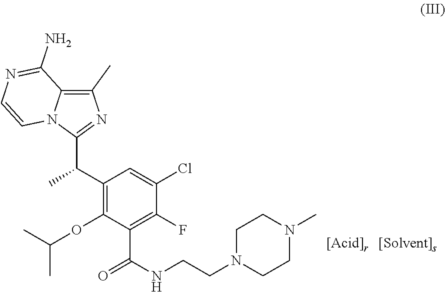 [SMILES: Cc1nc([C@@H](C)c2cc(Cl)c(F)c(C(=O)NCCN3CCN(C)CC3)c2OC(C)C)n2ccnc(N)c12]

wherein [Acid] is selected from the group consisting of organic acids and inorganic acids;
[Solvent] is selected from H₂O or organic solvents;
r is a number from about 0.0 to about 5.0;
s is a number from about 0.0 to about 5.0.
21. A crystalline form of Item 20, wherein [Acid] is selected from the group consisting of inorganic acid selected from Hydrochloric acid, sulfuric acid, phosphoric acid, hydrobromic acid and/or nitric acid; or organic acid selected from fumaric acid, tartaric acid (L-tartaric acid or d-tartaric acid), lauric acid, stearic acid, gentian acid, nicotinic acid, aspartic acid, succinic acid, adipic acid, malic acid (L-malic acid), citric acid, glycolic acid, gluconic acid (d-Gluconic acid), lactic acid (DL lactic acid), acetic acid, benzenesulfonic acid, methanesulfonic acid, methanesulfonic acid, benzoic acid, naphthalenesulfonic acid and/or oxalic acid;
preferably [Acid] is selected from sulfuric acid, fumaric acid, tartaric acid (L-tartaric acid or d-tartaric acid), sulfuric acid, lauric acid, stearic acid, gentian acid, nicotinic acid;
more preferably [Acid] is selected from fumaric acid.
22. A crystalline form of any one of Items 20-21, wherein r is a number about 0.0 to 3.0, preferably about 0.0 to 2.0, more preferably r is a number selected from the group consisting of 0.5±0.1, 1.0±0.2 and 1.5±0.2, even more preferably, r is 0.95˜1.05, 1.05˜1.15 or 1.45˜1.55; more preferably, r is 0.98˜1.02, 1.08˜1.12 or 1.48˜1.52; even more preferably, r is 1.0, 1.1 or 1.5.
23. A crystalline form of any one of Items 20-22, wherein the [solvent] is selected from MeOH, EtOH, i-PrOH, n-PrOH, n-BuOH, t-BuOH, acetone, butanone, pentanone, H₂O, MeCN, THF, ether, propyl ether, n-heptane, hexane, 1,4-dioxane, EtOAc.
24. A crystalline form of any one of Items 20-23, wherein s is a number about 0.0 to 3.0, preferably about 0.0 to 2.0, more preferably s is a number selected from the group consisting of 0.1±0.1, 0.5±0.1, 1.0±0.2, 1.5±0.2 and 2.0±0.2, even more preferably, s is 0˜0.2, 0.95˜1.05, 1.05˜1.15, 1.45˜1.55, 1.90˜2.10; more preferably, s is 0.98˜1.02, 1.08˜1.12 or 1.48˜1.52, 1.95˜2.15; even more preferably, s is 0, 0.1, 0.2, 1.0, 1.1, 1.5 or 2.0.
25. A crystalline form of Item 20, wherein the crystalline form is Formula IV

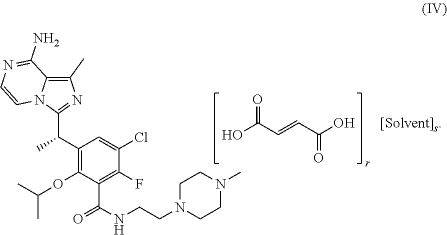

26. A crystalline form of Item 25, wherein the crystalline form is Formula V

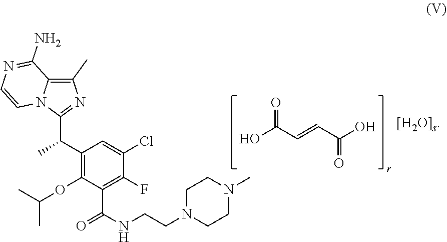

27. A crystalline form of Item 26, wherein r is a number about 0.0 to 3.0, preferably about 0.0 to 2.0, more preferably r is a number selected from the group consisting of 0.5±0.1, 1.0±0.2 and 1.5±0.2, even more preferably, r is 0.95˜1.05, 1.05˜1.15 or 1.45˜1.55; more preferably, r is 0.98˜1.02, 1.08˜1.12 or 1.48˜1.52; even more preferably, r is 1.0, 1.1 or 1.5; s is a number about 0.0 to 3.0, preferably about 0.0 to 2.0, more preferably s is a number selected from the group consisting of 0.1±0.1, 0.5±0.1, 1.0±0.2 and 1.5±0.2, even more preferably, s is 0˜0.2, 0.95˜1.05, 1.05˜1.15 or 1.45˜1.55; more preferably, s is 0.98˜1.02, 1.08˜1.12 or 1.48˜1.52; even more preferably, s is 0, 0.1, 0.2, 1.0, 1.1 or 1.5; even more preferably s is 0.
28. A crystalline form of any one of Items 20-27, which is selected from fumarate Crystalline Form A, characterized by a powder X-ray diffraction pattern comprising three, four, five, six, seven, eight, nine or more diffraction peaks having 2θ angle values independently selected from the group consisting of 8.69±0.2, 9.01±0.2, 10.11±0.2, 10.77±0.2, 13.48±0.2, 16.18±0.2, 16.80±0.2, 17.14±0.2, 17.74±0.2, 18.54±0.2, 19.69±0.2, 22.09±0.2, 23.37±0.2; or
fumarate Crystalline Form D, characterized by a powder X-ray diffraction pattern comprising three, four, five, six, seven, eight, nine or more diffraction peaks having 2θ angle values independently selected from the group consisting of 4.83±0.2, 7.92±0.2, 8.87±0.2, 9.64±0.2, 13.01±0.2, 14.07±0.2, 14.47±0.2, 17.75±0.2, 19.34±0.2, 20.24±0.2, 21.88±0.2, 22.72±0.2, 24.78±0.2, 26.20±0.2, 28.26±0.2, 29.60±0.2; or
fumarate Crystalline Form E, characterized by a powder X-ray diffraction pattern comprising three, four, five, six, seven, eight, nine or more diffraction peaks having 2θ angle values independently selected from the group consisting of 7.56±0.2, 8.93±0.2, 9.30±0.2, 10.73±0.2, 11.36±0.2, 12.00±0.2, 13.48±0.2, 13.99±0.2, 14.50±0.2, 15.93±0.2, 17.95±0.2, 18.70±0.2, 19.00±0.2, 20.22±0.2, 20.70±0.2, 21.28±0.2, 21.87±0.2, 22.78±0.2, 23.73±0.2, 24.20±0.2, 25.60±0.2, 26.29±0.2, 26.81±0.2, 28.21±0.2, 28.48±0.2; or
fumarate Crystalline Form F, characterized by a powder X-ray diffraction pattern comprising three, four, five, six, seven, eight, nine or more diffraction peaks having 2θ angle values independently selected from the group consisting of 4.60±0.2, 8.20±0.2, 9.16±0.2, 10.44±0.2, 12.06±0.2, 13.74±0.2, 14.55±0.2, 15.33±0.2, 15.86±0.2, 17.19±0.2, 18.33±0.2, 18.90±0.2, 19.42±0.2, 19.97±0.2, 20.96±0.2, 22.06±0.2, 22.45±0.2, 22.96±0.2, 23.33±0.2, 24.78±0.2; or
fumarate Crystalline Form G, characterized by a powder X-ray diffraction pattern comprising three, four, five, six, seven, eight, nine or more diffraction peaks having 2θ angle values independently selected from the group consisting of 7.06±0.2, 10.71±0.2; or
fumarate Crystalline Form H, characterized by a powder X-ray diffraction pattern comprising three, four, five, six, seven, eight, nine or more diffraction peaks having 2θ angle values independently selected from the group consisting of 8.13±0.2, 8.43±0.2, 9.37±0.2, 11.71±0.2, 12.21±0.2, 12.92±0.2, 15.69±0.2, 20.13±0.2, 22.15±0.2, 23.20±0.2; or
fumarate Crystalline Form I, characterized by a powder X-ray diffraction pattern comprising three, four, five, six, seven, eight, nine or more diffraction peaks having 2θ angle values independently selected from the group consisting of 8.74±0.2, 9.35±0.2, 10.80±0.2, 13.13±0.2, 13.99±0.2; or
fumarate Crystalline Form J, characterized by a powder X-ray diffraction pattern comprising three, four, five, six, seven, eight, nine or more diffraction peaks having 2θ angle values independently selected from the group consisting of 4.35±0.2, 7.61±0.2, 8.58±0.2, 10.08±0.2, 12.84±0.2, 13.33±0.2, 17.08±0.2, 20.26±0.2, 21.44±0.2, 22.73±0.2, 25.91±0.2, 30.18±0.2, 34.60, ±0.2; or
fumarate Crystalline Form K, characterized by a powder X-ray diffraction pattern comprising three, four, five, six, seven, eight, nine or more diffraction peaks having 2θ angle values independently selected from the group consisting of 4.87±0.2, 7.84±0.2, 8.90±0.2, 9.22±0.2, 9.58±0.2, 14.00±0.2, 14.69±0.2, 15.75±0.2, 17.82±0.2, 18.70±0.2, 19.02±0.2, 19.65±0.2, 20.06±0.2, 20.64±0.2, 21.21±0.2, 22.17±0.2, 22.98±0.2, 23.77±0.2, 24.65±0.2, 25.90±0.2, 26.85±0.2, 29.94±0.2, 32.08±0.2, 32.64±0.2, 33.48±0.2; or
fumarate Crystalline Form L, characterized by a powder X-ray diffraction pattern comprising three, four, five, six, seven, eight, nine or more diffraction peaks having 2θ angle values independently selected from the group consisting of 5.05±0.2, 7.89±0.2, 8.51±0.2, 10.11±0.2, 11.11±0.2, 13.98±0.2, 14.14±0.2, 15.16±0.2, 15.77±0.2, 17.15±0.2, 18.15±0.2, 18.43±0.2, 18.60±0.2, 19.86±0.2, 20.27±0.2, 20.96±0.2, 22.36±0.2, 22.69±0.2, 25.11±0.2, 25.43±0.2, 27.32±0.2, 28.54±0.2, 29.93±0.2, 30.60±0.2, 31.73±0.2, 33.26±0.2, 37.74±0.2, 38.76±0.2; or
fumarate Crystalline Form M, characterized by a powder X-ray diffraction pattern comprising three, four, five, six, seven, eight, nine or more diffraction peaks having 2θ angle values independently selected from the group consisting of 4.35±0.2, 8.65±0.2, 9.68±0.2, 10.69±0.2, 11.44±0.2, 12.96±0.2, 13.58±0.2, 14.28±0.2, 14.76±0.2, 15.52±0.2, 16.04±0.2, 16.67±0.2, 17.83±0.2, 18.41±0.2, 18.92±0.2, 19.18±0.2, 19.73±0.2, 20.25±0.2, 20.74±0.2, 21.04±0.2, 21.68±0.2, 22.09±0.2, 22.38±0.2, 22.65±0.2, 23.07±0.2, 23.41±0.2, 24.00±0.2, 24.69±0.2, 25.52±0.2, 26.01±0.2, 26.53±0.2, 27.81±0.2, 28.16±0.2, 28.76±0.2, 29.28±0.2, 29.77±0.2, 30.55±0.2, 30.79±0.2, 31.74±0.2, 31.99±0.2, 32.39±0.2, 33.46±0.2, 34.16±0.2, 34.43±0.2, 35.00±0.2, 35.77±0.2, 36.34±0.2, 36.81±0.2, 37.86±0.2, 38.56±0.2, 39.04±0.2, 39.55±0.2.
29. A crystalline form of any one of claims 20-27, which is selected from fumarate salt Type A, characterized by a powder X-ray diffraction pattern comprising diffraction peaks having 2θ angle values of 8.69±0.2, 9.01±0.2 and 10.77±0.2; preferably having 2θ angle values of 8.69±0.2, 9.01±0.2, 10.77±0.2, 16.8±0.2 and 17.14±0.2; more preferably having 2θ angle values of 8.69±0.2, 9.01±0.2, 10.77±0.2, 13.48±0.2, 16.8±0.2, 17.14±0.2 and 17.74±0.2; even more preferably having 2θ angle values of 8.69±0.2, 9.01±0.2, 10.11±0.2, 10.77±0.2, 13.48±0.2, 16.8±0.2, 17.14±0.2, 17.74±0.2 and 19.69±0.2; even more preferably having 2θ angle values of 8.69±0.2, 9.01±0.2, 10.11±0.2, 10.77±0.2, 13.48±0.2, 16.8±0.2, 17.14±0.2, 17.74±0.2, 19.69±0.2, 22.09±0.2 and 23.37±0.2; or
fumarate Type K, characterized by a powder X-ray diffraction pattern comprising diffraction peaks having 2θ angle values of 7.84±0.2, 14.69±0.2 and 15.75±0.2; preferably having 2θ angle values of 7.84±0.2, 8.9±0.2, 9.22±0.2, 14.69±0.2 and 15.75±0.2; more preferably having 2θ angle values of 7.84±0.2, 8.9±0.2, 9.22±0.2, 9.58±0.2, 14.69±0.2, 15.75±0.2 and 20.06±0.2; even more preferably having 2θ angle values of 7.84±0.2, 8.9±0.2, 9.22±0.2, 9.58±0.2, 14.69±0.2, 15.75±0.2, 19.65±0.2, 20.06±0.2 and 22.17±0.2; even more preferably having 2θ angle values of 7.84±0.2, 8.9±0.2, 9.22±0.2, 9.58±0.2, 14.69±0.2, 15.75±0.2, 18.7±0.2, 19.65±0.2, 20.06±0.2, 20.64±0.2 and 22.17±0.2; or
fumarate Type D, characterized by a powder X-ray diffraction pattern comprising diffraction peaks having 2θ angle values of 9.64±0.2, 14.47±0.2 and 19.34±0.2; preferably having 2θ angle values of 4.83±0.2, 9.64±0.2, 13.01±0.2, 14.47±0.2 and 19.34±0.2; more preferably having 2θ angle values of 4.83±0.2, 7.92±0.2, 9.64±0.2, 13.01±0.2, 14.07±0.2, 14.47±0.2 and 19.34±0.2; even more preferably having 2θ angle values of 4.83±0.2, 7.92±0.2, 9.64±0.2, 13.01±0.2, 14.07±0.2, 14.47±0.2, 17.75±0.2, 19.34±0.2 and 20.24±0.2; even more preferably having 2θ angle values of 4.83±0.2, 7.92±0.2, 8.87±0.2, 9.64±0.2, 13.01±0.2, 14.07±0.2, 14.47±0.2, 17.75±0.2, 19.34±0.2, 20.24±0.2 and 21.88±0.2; or
fumarate Type L, characterized by a powder X-ray diffraction pattern comprising diffraction peaks having 2θ angle values of 10.11±0.2, 15.16±0.2 and 20.27±0.2; preferably having 2θ angle values of 10.11±0.2, 13.98±0.2, 15.16±0.2, 20.27±0.2 and 22.69±0.2; more preferably having 2θ angle values of 10.11±0.2, 13.98±0.2, 14.14±0.2, 15.16±0.2, 18.6±0.2, 20.27±0.2 and 22.69±0.2; even more preferably having 2θ angle values of 7.89±0.2, 10.11±0.2, 13.98±0.2, 14.14±0.2, 15.16±0.2, 18.15±0.2, 18.6±0.2, 20.27±0.2 and 22.69±0.2; even more preferably having 2θ angle values of 7.89±0.2, 10.11±0.2, 13.98±0.2, 14.14±0.2, 15.16±0.2, 18.15±0.2, 18.43±0.2, 18.6±0.2, 19.86±0.2, 20.27±0.2 and 22.69±0.2; or
fumarate Type F, characterized by a powder X-ray diffraction pattern comprising diffraction peaks having 2θ angle values of 8.2±0.2, 9.16±0.2 and 13.74±0.2; preferably having 2θ angle values of 8.2±0.2, 9.16±0.2, 12.06±0.2, 13.74±0.2 and 18.33±0.2; more preferably having 2θ angle values of 4.6±0.2, 8.2±0.2, 9.16±0.2, 12.06±0.2, 13.74±0.2, 18.33±0.2 and 19.97±0.2; even more preferably having 2θ angle values of 4.6±0.2, 8.2±0.2, 9.16±0.2, 12.06±0.2, 13.74±0.2, 15.33±0.2, 18.33±0.2, 19.97±0.2 and 23.33±0.2; even more preferably having 2θ angle values of 4.6±0.2, 8.2±0.2, 9.16±0.2, 12.06±0.2, 13.74±0.2, 15.33±0.2, 18.33±0.2, 19.97±0.2, 20.96±0.2, 22.06±0.2 and 23.33±0.2; or
fumarate Type M, characterized by a powder X-ray diffraction pattern comprising diffraction peaks having 2θ angle values of 4.1±0.2, 6.83±0.2 and 10.23±0.2; preferably having 2θ angle values of 4.02±0.2, 4.1±0.2, 4.98±0.2, 6.83±0.2 and 10.23±0.2; more preferably having 2θ angle values of 3.21±0.2, 4.02±0.2, 4.1±0.2, 4.98±0.2, 6.52±0.2, 6.83±0.2 and 10.23±0.2; even more preferably having 2θ angle values of 3.21±0.2, 3.86±0.2, 4.02±0.2, 4.1±0.2, 4.22±0.2, 4.98±0.2, 6.52±0.2, 6.83±0.2 and 10.23±0.2; even more preferably having 2θ angle values of 3.21±0.2, 3.86±0.2, 4.02±0.2, 4.1±0.2, 4.22±0.2, 4.69±0.2, 4.98±0.2, 6.52±0.2, 6.83±0.2, 7.74±0.2 and 10.23±0.2; or
fumarate Type H, characterized by a powder X-ray diffraction pattern comprising diffraction peaks having 2θ angle values of 8.13±0.2, 8.43±0.2 and 9.37±0.2; preferably having 2θ angle values of 8.13±0.2, 8.43±0.2, 9.37±0.2, 12.92±0.2 and 22.15±0.2; more preferably having 2θ angle values of 8.13±0.2, 8.43±0.2, 9.37±0.2, 11.71±0.2, 12.92±0.2, 20.13±0.2 and 22.15±0.2; even more preferably having 2θ angle values of 8.13±0.2, 8.43±0.2, 9.37±0.2, 11.71±0.2, 12.21±0.2, 12.92±0.2, 20.13±0.2, 22.15±0.2 and 23.2±0.2; even more preferably having 2θ angle values of 8.13±0.2, 8.43±0.2, 9.37±0.2, 11.71±0.2, 12.21±0.2, 12.92±0.2, 15.69±0.2, 20.13±0.2, 22.15±10.2 and 23.2±0.2; or
fumarate Type J, characterized by a powder X-ray diffraction pattern comprising diffraction peaks having 2θ angle values of 4.35±0.2, 8.58±0.2 and 12.84±0.2; preferably having 2θ angle values of 4.35±0.2, 8.58±0.2, 12.84±0.2, 21.44±0.2 and 25.91±0.2; more preferably having 2θ angle values of 4.35±0.2, 7.61±0.2, 8.58±0.2, 10.08±0.2, 12.84±0.2, 21.44±0.2 and 25.91±0.2; even more preferably having 2θ angle values of 4.35±0.2, 7.61±0.2, 8.58±0.2, 10.08±0.2, 12.84±0.2, 20.26±0.2, 21.44±0.2, 22.73±0.2 and 25.91±0.2; even more preferably having 2θ angle values of 4.35±0.2, 7.61±0.2, 8.58±0.2, 10.08±0.2, 12.84±0.2, 13.33±0.2, 17.08±0.2, 20.26±0.2, 21.44±0.2, 22.73±0.2 and 25.91±0.2; or
fumarate Type E, characterized by a powder X-ray diffraction pattern comprising diffraction peaks having 2θ angle values of 8.93±0.2, 13.48±0.2 and 13.99±0.2; preferably having 2θ angle values of 8.93±0.2, 13.48±0.2, 13.99±0.2, 14.5±0.2 and 18.7±0.2; more preferably having 2θ angle values of 7.56±0.2, 8.93±0.2, 9.31±0.2, 13.48±0.2, 13.99±0.2, 14.5±0.2, 18.7±0.2 and 20.7±0.2; even more preferably having 2θ angle values of 8.93±0.2, 9.3±0.2, 13.48±0.2, 13.99±0.2, 14.5±0.2, 18.7±0.2, 19±0.2 and 20.7±0.2.
30. A crystalline form of any one of claims 20-27, substantially characterized by a powder X-ray diffraction pattern selected from the group consisting of FIGS. 2, 8, 10, 12, 14, 16, 18, 19, 20, 21, 25, 26, 30, 31, 34, 35, 38, 41, 42, 43, 44 and 45.
31. A pharmaceutical composition comprising a therapeutically effective amount of crystalline form according to any one of items 20-30, and optionally one or more pharmaceutically acceptable carrier(s).
32. A method for treating or preventing a disorder or a disease selected from inflammatory disorder, autoimmune disease, or a cancer, comprising administering a subject in need thereof a therapeutically effective amount of the crystalline form according to any one of items 20-30, or the pharmaceutical composition of item 31.
33. A process for the preparation of the crystalline form of item 28 or 29, comprising:
step (1) fumarate is dissolved in a mixture of EtOAc/MeOH, the clear solution is slow evaporated to give the crystalline; or
step (2) Fumarate is dissolved in EtOH, the solution is concentrated, the resulting material is stayed to give the crystalline; or
step (3) Fumarate is dissolved in EtOH, to the mixture is added n-heptane, the mixture is stirred to give the crystalline; or
step (4) Fumarate is placed in a water vapour atmosphere to give the crystalline; or
step (5) Fumarate is dissolved in a mixture of 1,4-dioxane and water, the mixture is stirred at room temperature and at −8° C.˜0° C. (preferably is −4° C.) to give the crystalline; or
step (6) Fumarate is dissolved in EtOH at 60° C.˜90° C. (preferably is 70° C.), the resulting clear solution is stirred at to give the crystalline; or
step (7) fumarate is dissolved in NMP, to the clear solution is added EtOAc, the resulting mixture is stirred to give the crystalline; or
step (8) Fumarate is placed in a EtOH vapour atmosphere to give the crystalline.
34. The process for the preparation of the crystalline form of item 33, wherein the time of step (1) is 5-10 days, preferably 7 days; and/or EtOAc/MeOH is 1:1 to 4:1, preferably is 2:1;
step (2) further comprises the solid is rinsed with EtOH and dried to give the crystalline;
the temperature of step (3) is room temperature and/or the time of step (3) is overnight;
the time of step (4) is 6-10 days, preferably is 8 days;
the ratio of 1,4-dioxane and water of step (5) is 8:1 to 10:1, preferably is 9/1;
the time of step (6) is 1-5 days, preferably is 2 days;
the temperature of step (7) is room temperature and/or the time of step (3) is overnight;
the time of step (8) is 6-10 days, preferably is 8 days; and/or step (8) comprises air-drying at RT overnight.
35. A process for the preparation of the crystalline form of item 28 or 29, comprising
step (a): a crystalline form is heated to 80˜160° C.; optionally further comprising
step (b): the crystalline form is cooled to 10˜40° C.
36. The process for the preparation of the crystalline form of item 35, wherein the crystalline form of step (a) is heated to 100˜150° C., preferably is 140° C.; the crystalline form of step (b) is cooled to 20˜35° C., preferably is 30° C.
37. The process for the preparation of the crystalline form of item 35, wherein process is under N₂ atmosphere.
38. The process for the preparation of the crystalline form of item 35, wherein the starting crystalline is selected from type A, D, F, G, H, J, E and I; preferably is type A, D, F.

### BRIEF DESCRIPTIONS OF THE DRAWINGS

FIG. 1 shows the ¹H-NMR spectrum for fumarate of Compound A(1.1:1)

FIG. 2 XRPD overlay of fumarate Type α batches

FIG. 3 TGA/DSC curves of fumarate Type α

FIG. 4 shows the ¹H-NMR spectrum for fumarate of Compound A(1.5:1).

FIG. 5 shows the ¹H-NMR spectrum for fumarate of Compound A(1:1).

FIG. 6 shows the ¹H-NMR spectrum for D-tartrate of Compound A(1.5:1).

FIG. 7 shows the ¹H-NMR spectrum for sulfate of Compound A.

FIG. 8 shows the XRPD pattern of sulfate Type A.

FIG. 9 shows the ¹H-NMR spectrum for laurate of Compound A.

FIG. 10 shows the XRPD pattern for laurate of Compound A.

FIG. 11 shows the ¹H-NMR spectrum for Stearate of Compound A.

FIG. 12 shows the XRPD pattern for Stearate of Compound A.

FIG. 13 shows the ¹H-NMR spectrum for Gentisate of Compound A

FIG. 14 shows the XRPD pattern for Gentisate of Compound A

FIG. 15 shows the ¹H-NMR spectrum for Nicotinate of Compound A(1.6:1).

FIG. 16 shows the XRPD pattern for Nicotinate of Compound A(1.6:1).

FIG. 17 shows the ¹H-NMR spectrum for Nicotinate of Compound A(1.4:1)

FIG. 18 shows the XRPD pattern for Nicotinate of Compound A(1.4:1)

FIG. 19 shows the XRPD pattern for Freebase type A

FIG. 20 shows the XRPD pattern for Freebase type B

FIG. 21 shows the XRPD pattern for fumarate Type D

FIG. 22 shows the ¹H NMR spectrum of fumarate Type D

FIG. 23 shows the TGA/DSC curves of fumarate Type D

FIG. 24 shows the DVS plot of fumarate Type D

FIG. 25 shows the VT-XRPD of fumarate Type D

FIG. 26 shows the XRPD overlay of three batches of fumarate Type A

FIG. 27 shows the ¹H NMR spectrum of fumarate Type A

FIG. 28 shows the TGA/DSC curves of fumarate Type A

FIG. 29 shows the DVS plot of fumarate Type A

FIG. 30 shows the VT-XRPD of fumarate Type A

FIG. 31 shows the XRPD overlay of three batches of fumarate Type F

FIG. 32 shows the ¹H NMR spectrum of fumarate Type F

FIG. 33 shows the TGA/DSC curves of fumarate Type F

FIG. 34 shows the VT-XRPD of fumarate Type F

FIG. 35 shows the XRPD pattern of fumarate Type G

FIG. 36 shows the ¹H NMR spectrum of fumarate Type G

FIG. 37 shows the TGA/DSC curves of fumarate Type G

FIG. 38 shows the XRPD pattern of fumarate Type H

FIG. 39 shows the ¹H NMR spectrum of fumarate Type H

FIG. 40 shows the TGA/DSC curves of fumarate Type H

FIG. 41 shows the XRPD overlay of fumarate Type J

FIG. 42 shows the XRPD pattern of fumarate Type E

FIG. 43 shows the XRPD pattern of fumarate Type I

FIG. 44 shows the XRPD overlay of fumarate Type D after storage for 1 week

FIG. 45 shows the XRPD overlay of fumarate Type F after storage for 1 week

FIG. 46 shows the XRPD of fumarate Type K

FIG. 47 shows the XRPD of fumarate Type L

FIG. 48 shows the XRPD of fumarate Type M

FIG. 49 shows the XRPD pattern of Compound A as the starting material.

### DETAILED DESCRIPTION OF THE INVENTION

Although a freebase may theoretically form pharmaceutically acceptable salts with many acids, Compound A as a specific freebase disclosed herein has been found cannot form a salt with many acids or cannot form a crystalline salt with the desired crystallinity. Among the many conventional acids or salt-forming agents including hydrochloric acid, sulfuric acid, phosphoric acid, L-tartaric acid, L-aspartic acid, maleic acid, fumaric acid, succinic acid, adipic acid, L-malic acid, citric acid, hippuric acid, L-ascorbic acid, acetic acid, glycolic acid, lauric acid, stearic acid, glutamic acid, D-gluconic acid, DL-lactic acid, benzenesulfonic acid, methanesulfonic acid, gentistic acid, oxalic acid, nicotinic acid. Among the acids (salt-forming agents), the inventors of the instant invention have found that fumaric acid is the only one that could form a crystalline with sharp peaks and a smooth baseline in the XRPD pattern. Inventors suprisingly found that fumarate of Compound A has a good crystallinity, safety and production compatibility.

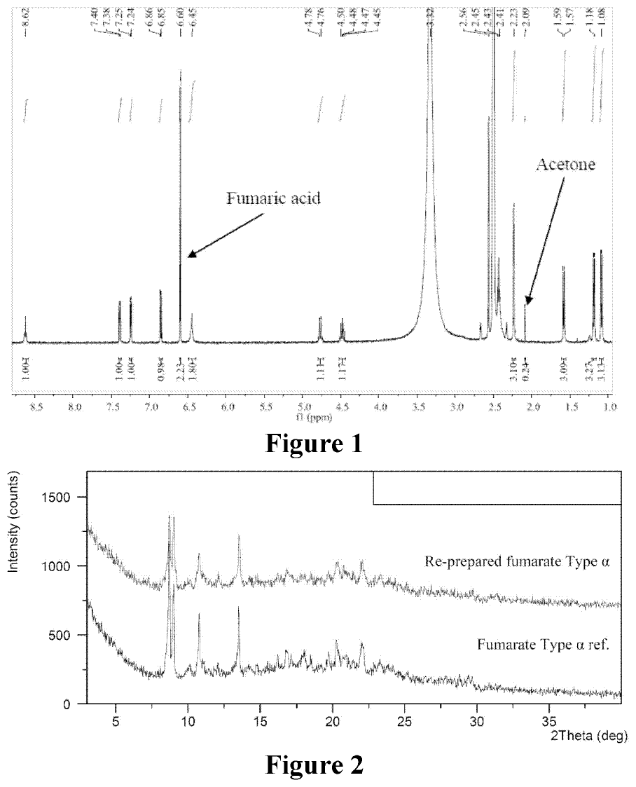

In one aspect, provided herein is the crystalline form of Compound A fumarate Type A. As shown in FIG. 1, the XRPD pattern thereof typically has the following peak diffraction angles (where “spacing” is shown as the “d-value” in FIG. 2):

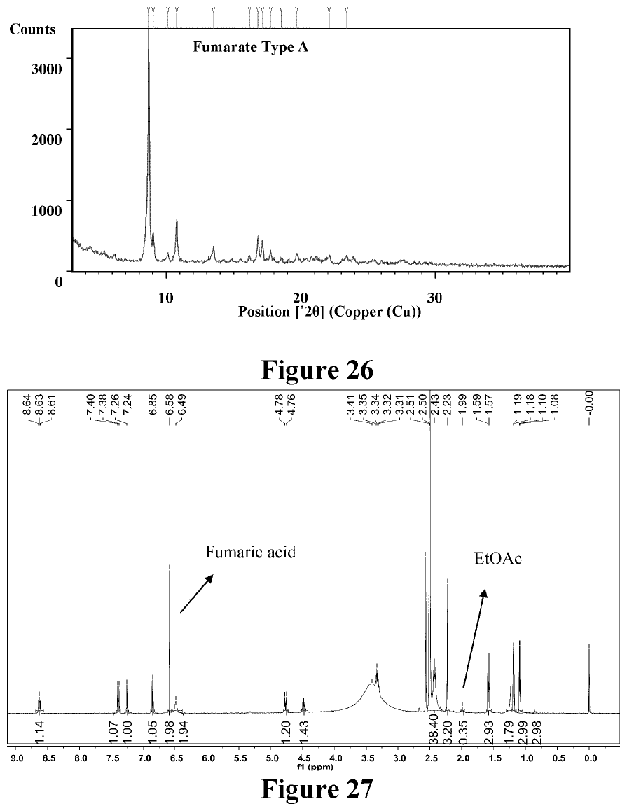

More specifically, the XRPD pattern of Compound A fumarate Type A has the following peak diffraction angles (where “spacing” is shown as the “d-value” in FIG. 26):

More specifically, the XRPD pattern thereof typically has the following peak diffraction angles (where “spacing” is shown as the “d-value” in FIG. 21):

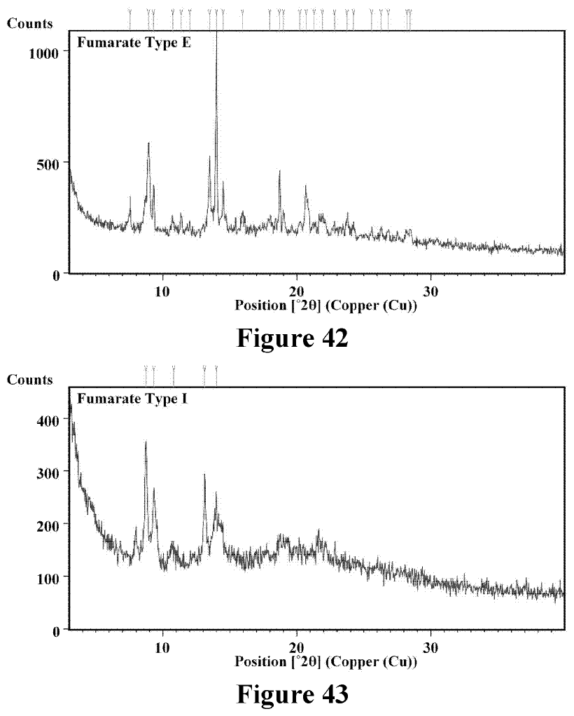

More specifically, the XRPD pattern of Compound A fumarate Type E typically has the following peak diffraction angles (where “spacing” is shown as the “d-value” in FIG. 42):

More specifically, the XRPD pattern thereof typically has the following peak diffraction angles (where “spacing” is shown as the “d-value” in FIG. 31):

More specifically, the XRPD pattern thereof typically has the following peak diffraction angles (where “spacing” is shown as the “d-value” in FIG. 35):

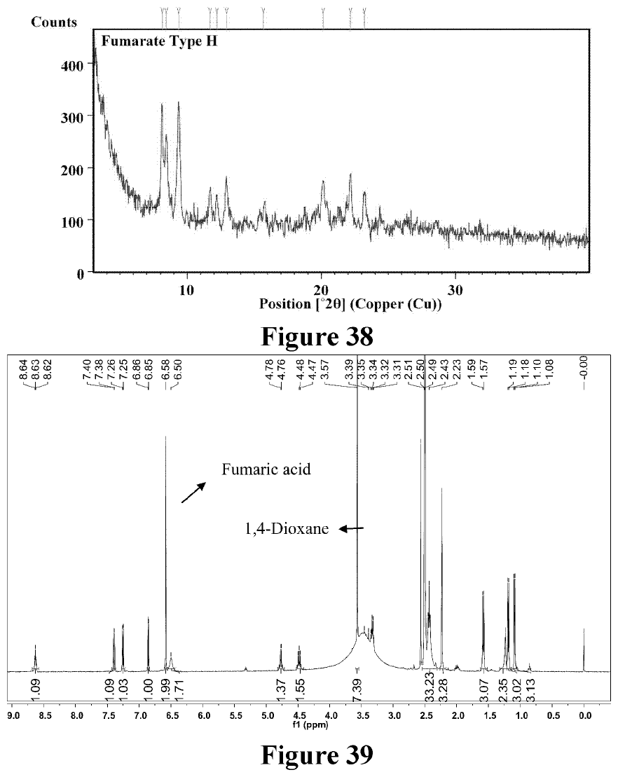

More specifically, the XRPD pattern of Compound A fumarate Type H typically has the following peak diffraction angles (where “spacing” is shown as the “d-value” in FIG. 38):

More specifically, the XRPD pattern thereof typically has the following peak diffraction angles (where “spacing” is shown as the “d-value” in FIG. 43):

More specifically, the XRPD pattern of Compound A fumarate Type J typically has the following peak diffraction angles (where “spacing” is shown as the “d-value” in FIG. 41):

More specifically, the XRPD pattern of Compound A fumarate Type K typically has the following peak diffraction angles (where “spacing” is shown as the “d-value” in FIG. 46):

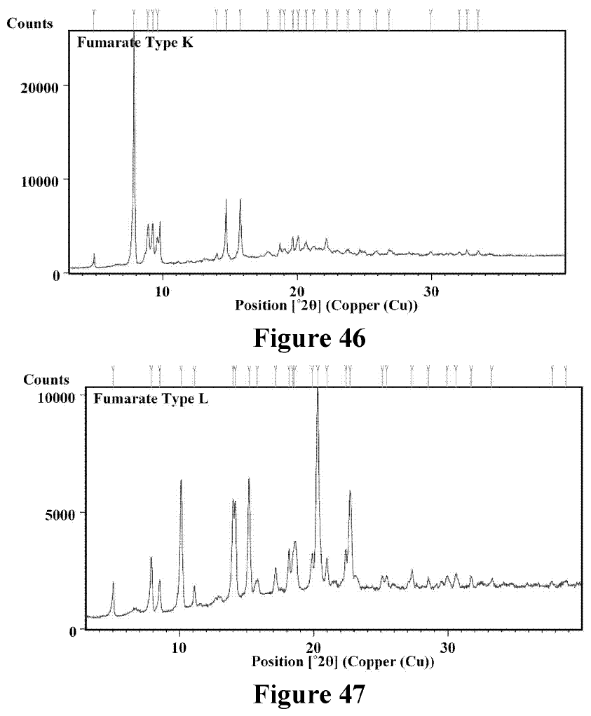

More specifically, the XRPD pattern of Compound A fumarate Type L typically has the following peak diffraction angles (where “spacing” is shown as the “d-value” in FIG. 47):

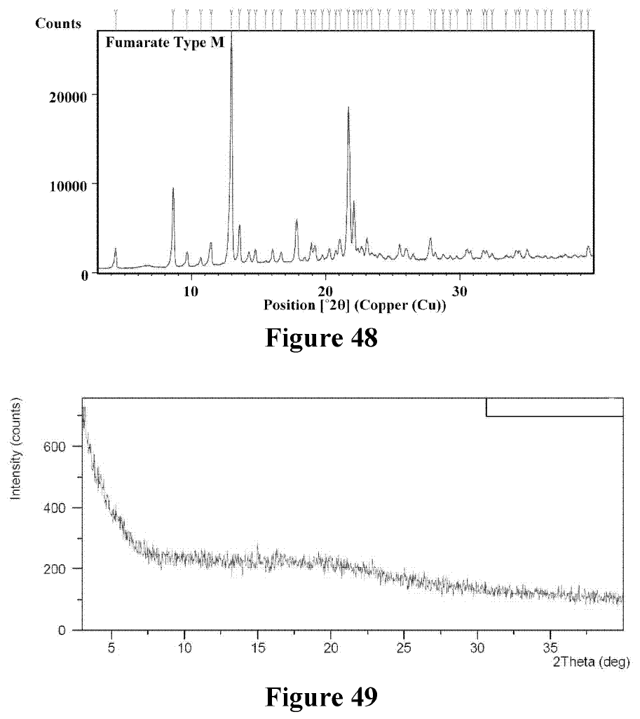

More specifically, the XRPD pattern of Compound A fumarate Type M typically has the following peak diffraction angles (where “spacing” is shown as the “d-value” in FIG. 48):

In one aspect, provided herein is the crystalline form of Compound A fumarate Type F. As shown in FIG. 31, the XRPD pattern thereof typically has the following peak diffraction angles (where “spacing” is shown as the “d-value” in FIG. 31):

The crystalline forms described above are rather stable crystalline forms.

For crystalline forms described above, only the main peaks (i.e., the most characteristic, significant, unique and/or reproducible peaks) are summarized; additional peaks may be obtained from the diffraction spectra by conventional methods. The main peaks described above can be reproduced within the margin of error (+ or −2 at the last given decimal place, or + or −0.2 at the stated value).

The method for preparing the free base of Compound A is disclosed in WO2019/047915A1. For the above-mentioned crystalline forms, the crystallization step can be conducted in an appropriate solvent system containing at least one solvent by evaporation of solvent, cooling and/or by addition of anti-solvents (solvents that are less able to solubilize the Compound A or its salts, including but not limited to those described herein) to achieve super-saturation in the solvent system.

Crystallization may be done with or without seed crystals, which is described in the present invention.

In an embodiment in this aspect, provided herewith is the fumarate of Compound A, preferably in the above-mentioned crystalline forms, more preferably in the crystalline forms of Types B, C, D and F, even more preferably in the crystalline forms of Types D and F, most preferably in the crystalline form of Type D.

The individual crystalline forms provided by the present invention develop under specific conditions dependent on the particular thermodynamic and equilibrium properties of the crystallization process. Therefore, a person skilled in the art will know that the crystals formed are a consequence of the kinetic and thermodynamic properties of the crystallization process. Under certain conditions (such as in a specific solvent), a particular crystalline form may have better properties than another crystalline form (or in fact have better properties than any other crystalline forms).

In another aspect, provided herein is a pharmaceutical composition each containing an effective amount of fumarate of Compound A, preferably in any of the above-described crystalline forms. The active compound can be 1-99% (by weight), preferably 1-70% (by weight), or more preferably 1-50% (by weight), or most preferably, 5-40% (by weight), of the composition.

In another aspect, provided herein is the use of the above-described salt or crystalline forms of Compound A in the manufacture of medicaments for the treatment of a cancer associated with PI3K delta inhibition.

In another aspect, provided herein is a pharmaceutical composition each containing an effective amount of fumarate salt of Compound A, preferably in any of the above-described crystalline forms, more preferably fumarate salt type D. The active compound can be 1-99% (by weight), preferably 1-70% (by weight), or more preferably 1-50% (by weight), or most preferably, 5-40% (by weight), of the composition.

The term “about” as used herein, unless indicated otherwise, denotes that a number (e.g., temperature, pH, volume, etc.) can vary within ±10%, preferably within ±5%.

A solvate herein is defined as a compound formed by solvation, for example as a combination of solvent molecules with molecules or ions of a solute. The known solvent molecules include water, alcohols and other polar organic solvents. Alcohols include methanol, ethanol, n-propanol, iso-propanol, n-butanol, iso-butanol and t-butanol. The preferred solvent is typically water. The solvate compounds formed by solvation with water are sometimes termed as hydrates.

In some embodiments, the crystalline form has a crystalline purity at least about 80%, preferably at least about 90%, preferably at least about 95% crystalline purity, preferably about 97% crystalline purity, more preferably about 99% or more crystalline purity, and most preferably about 100% crystalline purity.

The term “crystalline purity,” as used herein, means the percentage of a particular crystalline form of a compound in a sample, which may contain the amorphous form of the compound, one or more other crystalline forms of the compound (other than the particular crystalline form of the compound), or a mixture thereof. Crystalline purity is determined by X-ray powder diffraction (XRPD), Infrared Raman spectroscopy and other solid state methods.

The following synthetic methods, specific examples, and efficacy tests further describe certain aspects of the present invention. They shall not limit or restrict the scope of the present invention in any way.

### EXAMPLES

### Example 1: Preparation of Free Base of Compound A ((S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide)

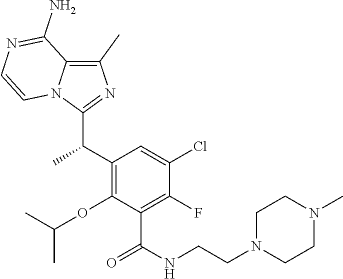 [SMILES: Cc1nc([C@@H](C)c2cc(Cl)c(F)c(C(=O)NCCN3CCN(C)CC3)c2OC(C)C)n2ccnc(N)c12]

To a solution of (S)-3-(1-(8-amino-1-methylimidazo[1,5-a]pyrazin-3-yl)ethyl)-5-chloro-6-fluoro-2-isopropoxybenzoic acid (20 g, 49.2 mmol) in dichloromethane (100 mL) was added SOCl₂ (29 g, 244 mmol) dropwise. The mixture was stirred at room temperature overnight. The mixture was concentrated under vacuum. The residue was dissolved in dichloromethane (200 mL). To the solution was added N-ethyl-N-isopropylpropan-2-amine (19 g, 147 mmol) at 0° C., and then a solution of 2-(4-methylpiperazin-1-yl)ethan-1-amine HCl salt (10.5 g, 70.3 mmol) in DCM (20 mL) was added dropwise. The mixture was stirred at 0° C. for 2 hours. The mixture was diluted with water (200 mL), extracted with dichloromethane (3×200 mL). The organic layers were combined, dried over Na₂SO₄, filtered and concentrated. The residue was purified by silica gel column chromatography (eluent with dichloromethane:MeOH:ammonia water=100:10:0.5) to give the title compound (7.2 g, 27%). LC-MS (M+H)=531.9.

¹H NMR (400 MHz, dmso) δ 8.63 (t, J=5.7 Hz, 1H), 7.38 (d, J=8.6 Hz, 1H), 7.25 (d, J=5.0 Hz, 1H), 6.85 (d, J=5.0 Hz, 1H), 6.43 (brs, 2H), 4.77 (q, J=6.9 Hz, 1H), 4.52-4.45 (m, 1H), 3.36-3.29 (m, 2H), 2.56 (s, 3H), 2.46-2.26 (m, 10H), 2.16 (s, 3H), 1.58 (d, J=7.1 Hz, 3H), 1.19 (d, J=6.0 Hz, 3H), 1.09 (d, J=6.0 Hz, 3H).

### Example 2: Formation of Salts

Salt formations were performed using each of 25 acids (HCl, H2SO4, H3PO4, L-tartaric acid, L-aspartic acid, Maleic acid, Fumaric acid, Succinic acid, Adipic acid, L-malic acid, Citric acid, Hippuric acid, L-ascorbic acid, Acetic acid, Glycolic acid, Laurie acid, Stearic acid, Glutamic acid, D-gluconic acid, DL-Lactic acid, Benzenesulfonic acid, Methanesulfonic acid, Gentistic acid, Oxalic acid, Nicotinic acid) as well as blank as the control in four solvent systems (Solvent: A was IPA/n-heptane (1:4, v/v); B was acetone/n-heptane (1:4, v/v); C was IPAc/n-heptane (4:1, v/v); D was 1,4-dioxane) via solvent-assisted reaction crystallization. In detail, about 15 mg of amorphous freebase (Compound A) and corresponding acid were mixed into each HPLC vial with the desired molar ratio of 1:1. 0.3 mL of the corresponding solvent was then added to form a suspension, which was magnetically stirred (˜800 rpm) at RT for about three days. Solids were isolated for XRPD analysis. The results are summarized in Table 1.

TABLE 1
Results of Salt Formation

| | | Solvent | | | |
|---|---|---|---|---|---|
| Acid | | A | B | C | D |
| 0 | Blank | Amorphous | Amorphous | Amorphous | Freebase Type A |
| | | Freebase | Freebase | Freebase | |
| 1 | HCl | Amorphous Salt | Amorphous Salt | Amorphous Salt | Freebase Type A |
| 2 | H₂SO₄ | Sulfate Type A | Amorphous Salt | Amorphous Salt | Amorphous Salt |
| 3 | H₃PO₄ | Amorphous Salt | Amorphous Salt | Amorphous Salt | Amorphous Salt |
| 4 | L-tartaric acid | Acid | Amorphous Salt | Acid | Amorphous Salt |
| 5 | L-aspartic acid | Acid | Acid | Acid | Acid |
| 6 | Maleic acid | Amorphous Salt | Acid | Amorphous Salt | Amorphous Salt |
| 7 | Fumaric acid | Acid | Fumarate Type A | Acid | Amorphous Salt |
| 8 | Succinic acid | Amorphous Salt | Amorphous Salt | Acid | Freebase Type A |
| 9 | Adipic acid | Amorphous Salt | Amorphous Salt | Amorphous Salt | Amorphous Salt |
| 10 | L-malic acid | Amorphous Salt | Amorphous Salt | Amorphous Salt | Freebase Type A |
| 11 | Citric acid | Amorphous Salt | Amorphous Salt | Acid | Freebase Type A |
| 12 | Hippuric acid | Freebase Type B | Freebase Type B | Freebase Type B + | Freebase Type B |
| | | | | Acid | |
| 13 | L-ascorbic acid | 2 peaks | Amorphous Salt | Acid | Freebase Type B |
| 14 | Acetic acid | Amorphous Salt | Amorphous Salt | Amorphous Salt | Amorphous Salt |
| 15 | Glycolic acid | Amorphous Salt | Amorphous Salt | Acid | Freebase Type A + |
| | | | | | Acid |
| 16 | Lauric acid | 2 peaks | Amorphous Salt | Laurate Type A | Amorphous Salt |
| 17 | Stearic acid | Stearate Type A | Amorphous Salt | Stearate Type A | Freebase Type A |
| 18 | Glutamic acid | Acid | Acid | Acid | Acid |
| 19 | D-gluconic acid | Amorphous Salt | Amorphous Salt | Amorphous Salt | Freebase Type A |
| 20 | DL-Lactic acid | Amorphous Salt | Amorphous Salt | Amorphous Salt | Freebase Type A |
| 21 | Benzenesulfonic | 1 peak | 1 peak | 1 peak | Freebase Type A |
| | acid | | | | |
| 22 | Methanesulfonic | 3 peaks | Amorphous Salt | 1 peak | 1 peak |
| | acid | | | | |
| 23 | Gentistic acid | Amorphous Salt | Amorphous Salt | Amorphous Salt | Gentisate Type A |
| 24 | Oxalic acid | Amorphous Salt | Amorphous Salt | Amorphous Salt | Amorphous Salt |
| 25 | Nicotinic acid | Nicotinate Type A | Nicotinate Type B | Acid | Amorphous Salt |

Solvent: A was IPA/n-heptane (1:4, v/v); B was acetone/n-heptane (1:4, v/v); C was IPAc/n-heptane (4:1, v/v); D was 1,4-dioxane.

As summarized in Table 1, a total of seven potential crystalline salts (sulfate Type A, fumarate Type A, laurate Type A, stearate Type A, gentisate Type A, nicotinate Type A and nicotinate Type B) and two freebases (freebase Type A and B) were observed based on the XRPD comparison, wherein the two freebases (freebase Type A and B) were obtained as either in an amorphous form or in a gel. Another two crystalline salts (fumarate Type B and fumarate Type C) were obtained in the re-preparation process. The other experiments gave either amorphous salts or acids (indicating that no salt has been formed).

### Example 3: Preparation of Fumarate Type α

15.01 mg the free base of Compound A and 3.28 mg of fumaric acid were mixed into a vial. 0.3 mL acetone/n-heptane (1:4. V/V) was added to form a suspension. The suspension is stirred at room temperature at 800 rpm for 2 days and transferred to slurry at 5° C. at 800 rpm for another 2 days. The fumarate product was isolated by centrifugation and vacuum dried at room temperature for 3 days to obtain fumarate of Compound A.

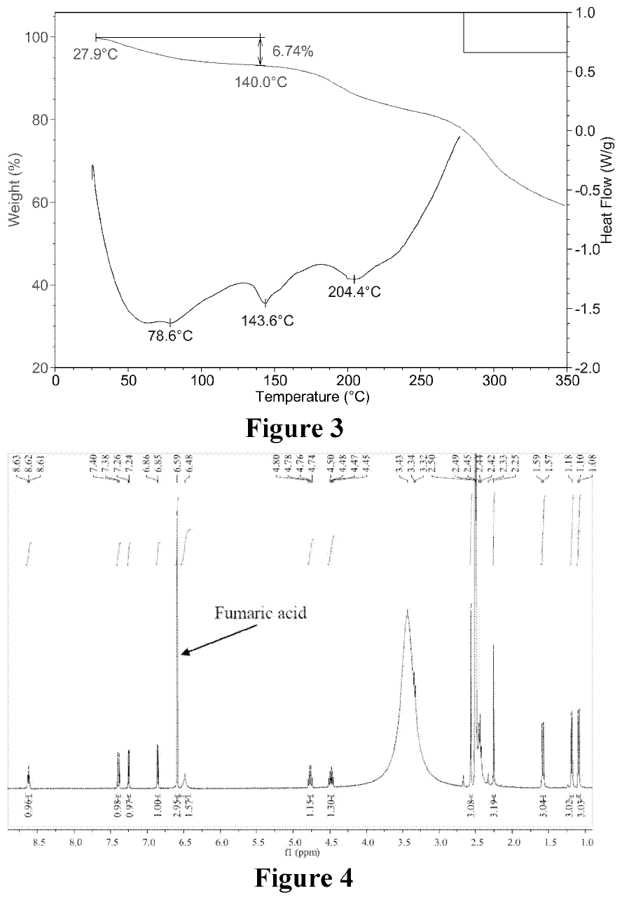

Two batches of fumarate Type α were obtained via slurry of equimolar amorphous freebase and fumaric acid in acetone/n-heptane (1:4, v/v) at RT and then vacuum drying at RT. XRPD patterns were displayed in FIG. 2. TGA/DSC and ¹H NMR (Bruker 400M NMR Spectrometer using DMSO-d₆) results of fumarate Type α were displayed in FIG. 3 and FIG. 1. A weight loss of 6.7% up to 140° C. was observed on TGA curve. DSC curve showed three endotherms at 78.6, 143.6 and 204.4° C. (peak). The molar ratio of acid/base was 1.1:1 and residual solvent acetone/API was 0.04:1 (0.4 wt %).

### Example 4: Preparation of Fumarate Type β

To a solution of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (1.0 g, freebase of Compound A) in EtOH (2 mL) was added a solution of fumaric acid (220 mg) in EtOH (4 mL). The mixture was stirred for 10 minutes. Then to the mixture was added n-butanol (6 mL). The resulting mixture was stirred at room temperature for 72 hours, then the product was obtained. ¹H NMR spectra were was collected on Bruker 400M NMR Spectrometer using DMSO-d₆. ¹H NMR spectrum showed the molar ratio of acid/free base was 1.5:1 (FIG. 4).

### Example 5: Preparation of Fumarate Type γ

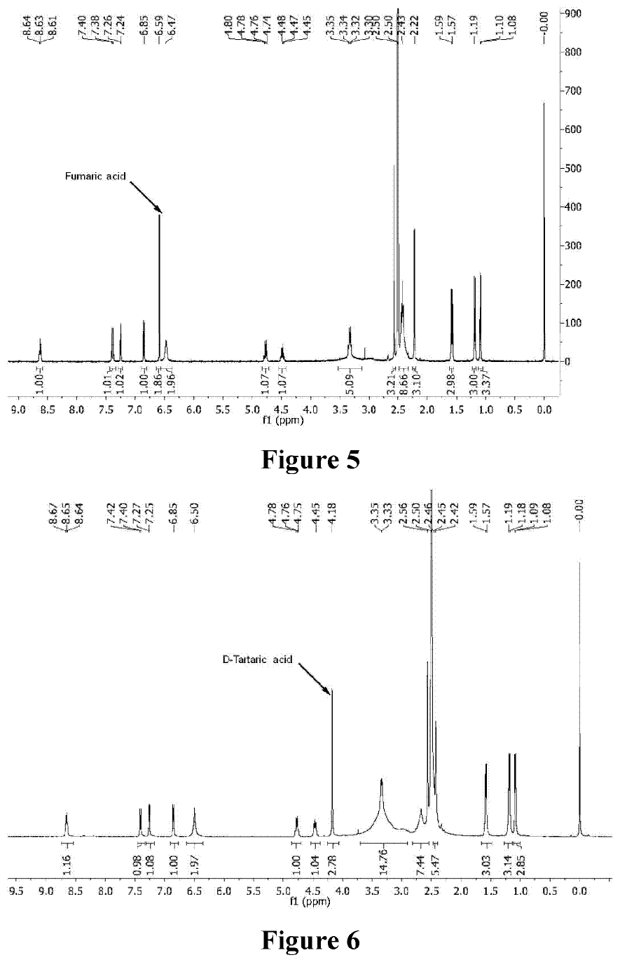

To a solution of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (5.0 g, the free base of Compound A) in EtOH (30 mL) was added a solution of fumaric acid (970 mg) in EtOH (50 mL). The mixture was stirred for 30 minutes. Then to the mixture was concentrated until about 24 g residue in the bottom. The resulting mixture was stirred at room temperature overnight, then the product was obtained. ¹H NMR spectrum showed the molar ratio of acid/free base was 1:1 (FIG. 5). ¹H NMR (400 MHz, dmso) δ 8.63 (t, J=5.5 Hz, 1H), 7.39 (d, J=8.6 Hz, 1H), 7.25 (d, J=5.1 Hz, 1H), 6.85 (d, J=5.0 Hz, 1H), 6.59 (s, 2H), 6.47 (brs, 2H), 4.77 (q, J=7.2 Hz, 1H), 4.52-4.44 (m, 1H), 3.33 q, J=6.3 Hz, 2H), 2.56 (s, 3H), 2.47-2.35 (m, 8H), 2.22 (s, 3H), 1.58 (d, J=7.0 Hz, 3H), 1.19 (d, J=6.0 Hz, 3H), 1.13-1.05 (m, 3H).

### Example 6: Preparation of D-tartrate

300 mg the free base of Compound A and 93 mg of D-tartaric acid was mixed into a vial with EtOH (10 mL), which was magnetically stirred at room temperature for about 30 min, then the product was obtained. ¹H NMR (400 MHz, DMSO) δ 8.65 (t, J=5.3 Hz, 1H), 7.41 (d, J=8.5 Hz, 1H), 7.26 (d, J=5.0 Hz, 1H), 6.86 (d, J=4.9 Hz, 1H), 6.50 (brs, 2H), 4.77 (q, J=6.7 Hz, 1H), 4.51-4.41 (m, 1H), 4.18 (s, 3H), 3.70-2.90 (m, 11H), 2.75-2.55 (m, 7H), 2.47-2.40 (m, 6H), 1.58 (d, J=7.0 Hz, 3H), 1.19 (d, J=5.9 Hz, 3H), 1.09 (d, J=5.9 Hz, 3H). ¹H NMR spectrum showed the molar ratio of acid/freebase was 1.5:1 (FIG. 6).

### Example 7: Preparation of Sulfate Type A

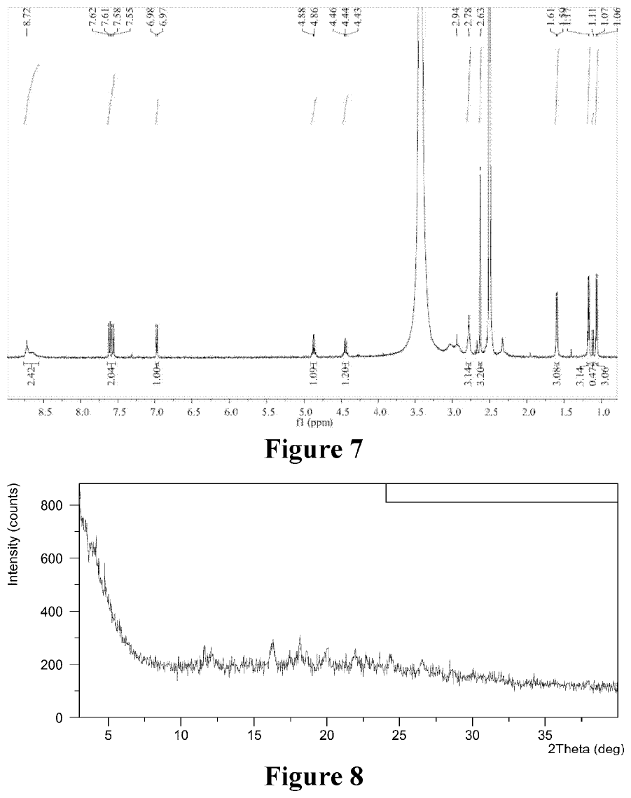

Sulfate of Compound A was obtained via slurry of the equimolar free base of Compound A and sulfuric acid in isopropyl alcohol/n-heptane (1:4, v/v) at room temperature and then vacuum drying at room temperature. ¹H NMR was shown in FIG. 7. XRPD pattern was shown in FIG. 8.

### Example 8: Preparation of Laurate Type A

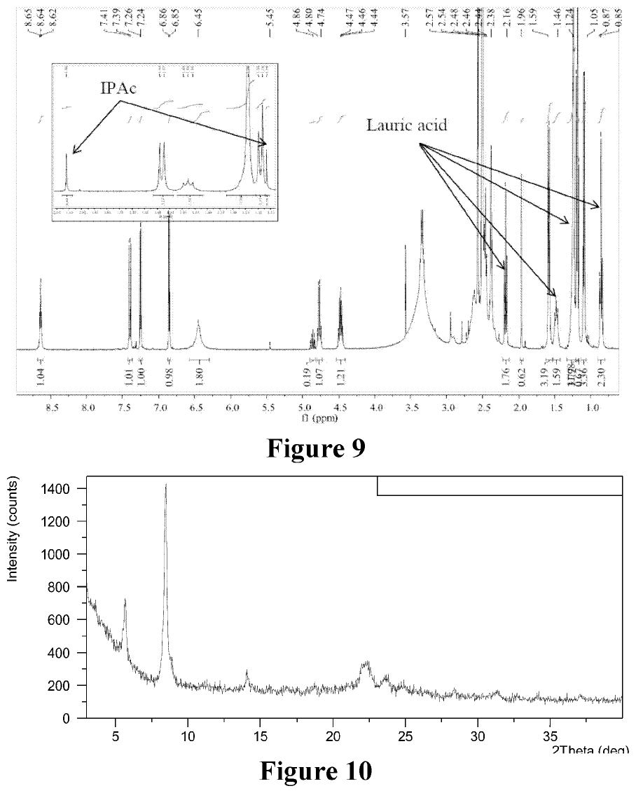

Laurate of Compound A was obtained via slurry of the equimolar free base of Compound A and lauric acid in isopropyl acetate/n-heptane (4:1, v/v) at room temperature and then vacuum drying at room temperature. ¹H NMR in FIG. 9 showed the molar ratio of acid/free base was 0.8:1. XRPD pattern was shown in FIG. 10.

### Example 9: Preparation of Stearate Type A

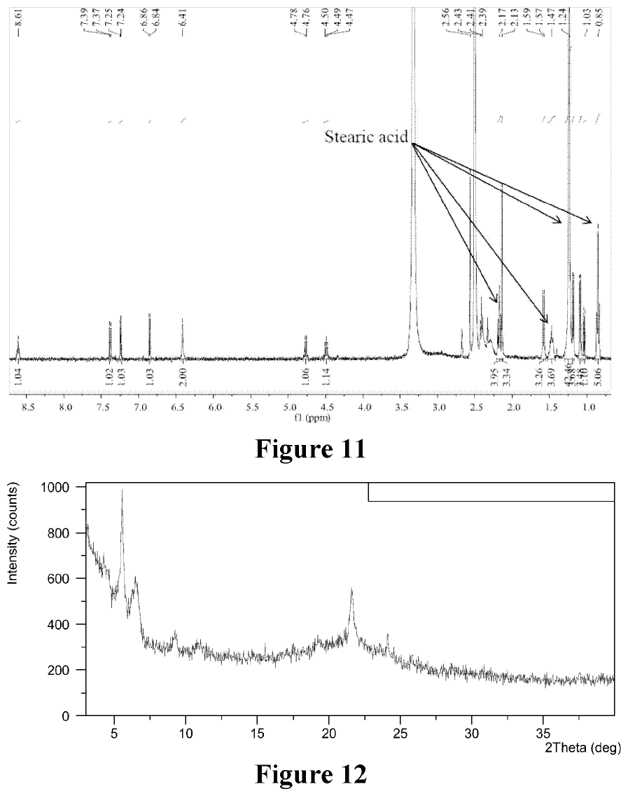

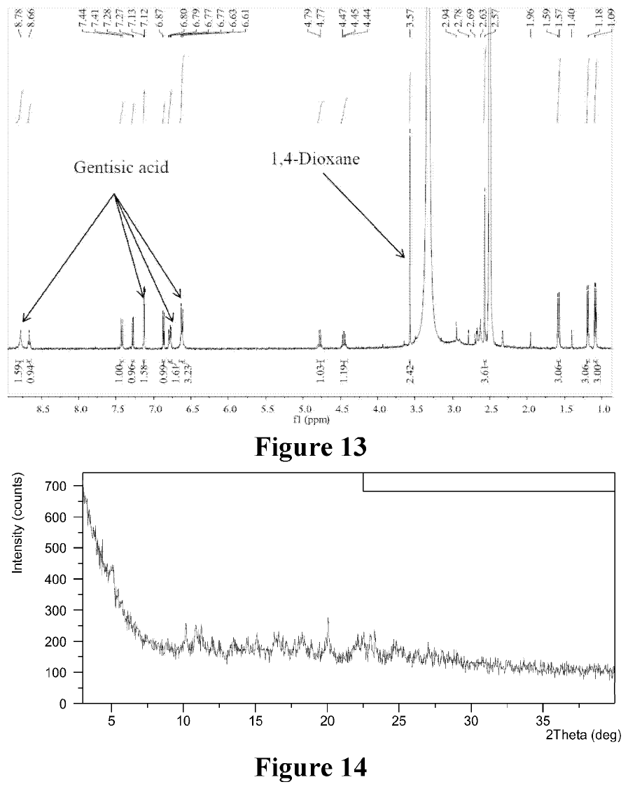

Stearate of Compound A was obtained via slurry of the equimolar free base of Compound A and stearic acid in isopropyl alcohol/n-heptane (1:4, v/v) at room temperature and then vacuum drying at room temperature. ¹H NMR in FIG. 11 showed the molar ratio of acid/free base was 1.8:1 and isopropyl alcohol or n-heptane was not detected. XRPD pattern was shown in FIG. 12.

### Example 10: Preparation of Gentisate Type A

Gentisate of Compound A was obtained via slurry of the equimolar free base of Compound A and gentisic acid in 1,4-dioxane at room temperature and then vacuum drying at room temperature. ¹H NMR in FIG. 8 showed the molar ratio of acid/free base was 1.6:1. XRPD pattern was shown in FIG. 14.

### Example 11: Preparation of Nicotinate Type A

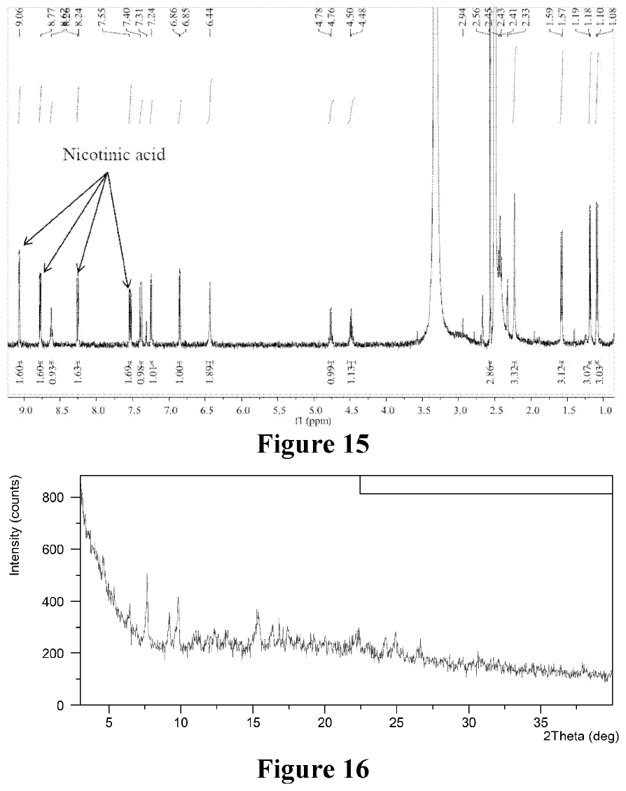

Nicotinate of Compound A was obtained via slurry of the equimolar free base of Compound A and nicotinic acid in isopropyl alcohol/n-heptane (1:4, v/v) at room temperature and then vacuum drying at room temperature. ¹H NMR in FIG. 15 showed the molar ratio of acid/free base was 1.6:1. XRPD pattern was shown in FIG. 16.

### Example 12: Preparation of Nicotinate Type B

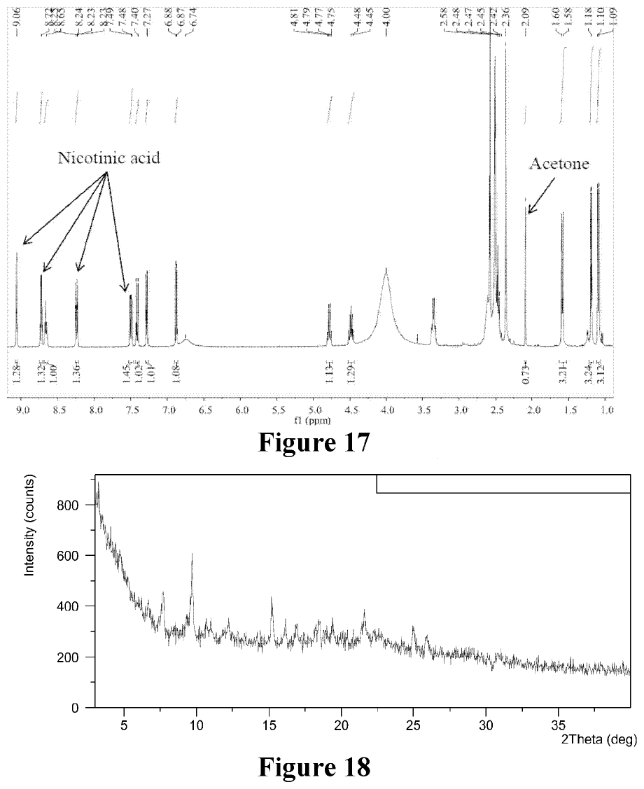

Nicotinate of Compound A was obtained via slurry of the equimolar free base of Compound A and nicotinic acid in acetone/n-heptane (1:4, v/v) at room temperature and then vacuum drying at room temperature. ¹H NMR in FIG. 17 showed the molar ratio of acid/free base was 1.4:1. XRPD pattern was shown in FIG. 18.

### Example 13: Preparation of Freebase Type A

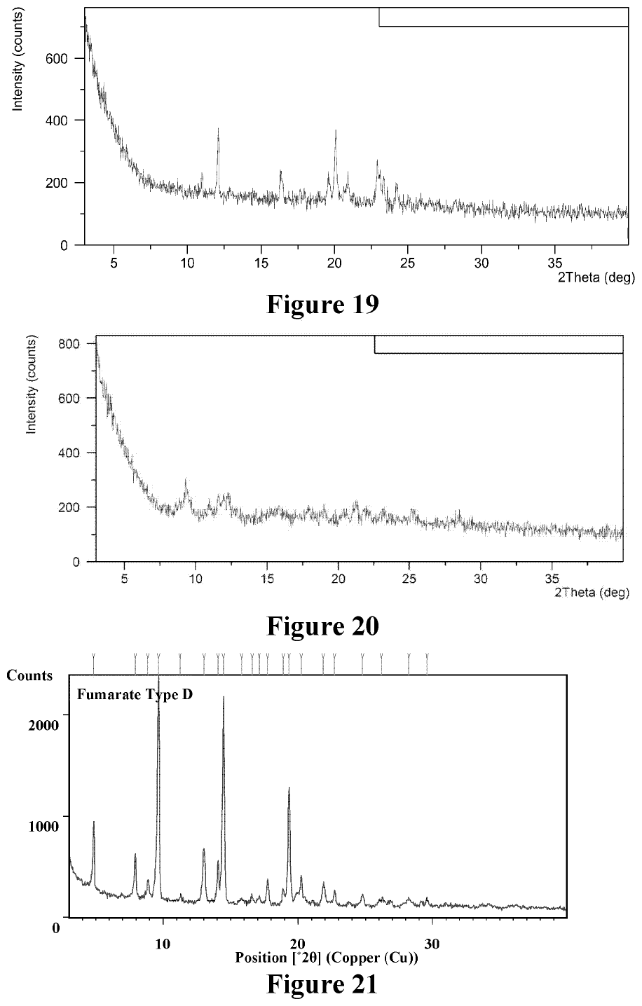

Freebase type A was obtained via slurry of amorphous freebase (Compound A) in 1,4-dioxane at RT. XRPD pattern was shown in FIG. 19 showing very low crystallinity.

### Example 14: Preparation of Freebase Type B

Freebase type B was obtained via slurry of equimolar amorphous freebase (Compound A) and hippuric acid in 1,4-dioxane at RT XRPD pattern was shown in FIG. 20 showing very low crystallinity.

### Example 15: Preparation of Other Crystalline Forms of the Fumarate

Upon our findings that fumarate is the only salt that could potentially form a crystalline, the development of further crystalline is performed using different crystallization or solid transition methods, including anti-solvent addition, liquid vapor diffusion, solid vapor diffusion, slow evaporation, slurry conversion at RT, slurry conversion at 50° C., temperature cycling, polymer induced crystallization, and etc. In the above methods, DMSO, NMP, MeOH, EtOH, water, toluene, THF, 2-MeTHF, MEK, MIBK, MTBE, EtOAc, DCM, anisole, IPA, IPAc, n-heptane, ACN, acetone, butyl acetate, CHCl3, 1,4-dioxane and the mixture thereof are used as the solvent and/or anti-solvent. Types A, D, E, F, G, H, I, J, K, L and M are prepared in the processes specified below.

Experiments were performed using compound A fumarate (1:1) as the starting material. A total of 11 crystal forms were obtained and characterized by X-ray powder diffraction (XRPD), thermo gravimetric analysis (TGA), differential scanning calorimetry (DSC), and solution proton nuclear magnetic resonance (¹H NMR). Further form identification study confirmed that among the 11 crystal forms, there are three hydrates (fumarate Type A, Type F and Type G), one anhydrate (fumarate Type D), three metastable anhydrates (fumarate Type K, Type L and Type M), two solvates (fumarate Type H and Type J) and two to be identified forms (fumarate Type E and Type I) that were challenging to re-prepare. Characterization summary for all the crystal forms was presented in Table 2.

TABLE 2
Characterization summary for fumarate crystal forms

| Crystal | Weightloss | Endotherm | | |
|---|---|---|---|---|
| form of | in TGA, | inDSC, ° C. | Stoichiometry | |
| fumarate | wt % | (peak) | (acid:freebase) | Solid form |
| Fumarate | 3.5 | 126.3, 162.8 | 1.0 | anhydrate |
| Type D | | | | |
| Fumarate | 9.1 | 88.9, 114.5, | 0.99 | Hydrate |
| Type A | | 173.5 | | |
| Fumarate | 8.8 | 92.9, 112.6, | 0.97 | Hydrate |
| Type F | | 189.9 | | |
| Fumarate | 18.6 | 80.6, 117.0, | 0.98 | Hydrate |
| Type G | | 153.5 | | |
| Fumarate | NA | NA | NA | Metastable |
| Type L | | | | anhydrate |
| Fumarate | NA | NA | NA | Metastable |
| Type K | | | | anhydrate |
| Fumarate | NA | NA | NA | Metastable |
| Type M | | | | anhydrate |
| Fumarate | NA | NA | NA | EtOH |
| Type J | | | | solvate |
| Fumarate | 14.4 | 76.2, 87.8, | 1.0 | Solvate |
| Type H | | 106.5, 187.0 | | |
| Fumarate | NA | NA | NA | Unidentified |
| Type E | | | | |
| Fumarate | NA | NA | NA | Unidentified |
| Type I | | | | |

### Example 16. Fumarate Type A and Type K

Crystalline form of fumarate Type A was obtained via the following procedure: fumarate (20.7 mg) was dissolved in a mixture of EtOAc/MeOH (2:1, v/v, 0.6 mL). The clear solution was stayed in a quiet place and slow evaporated for 7 days to give fumarate type A.

Type K was obtained via heated Type A to 140° C. under nitrogen atmosphere, and cooled to 30° C.

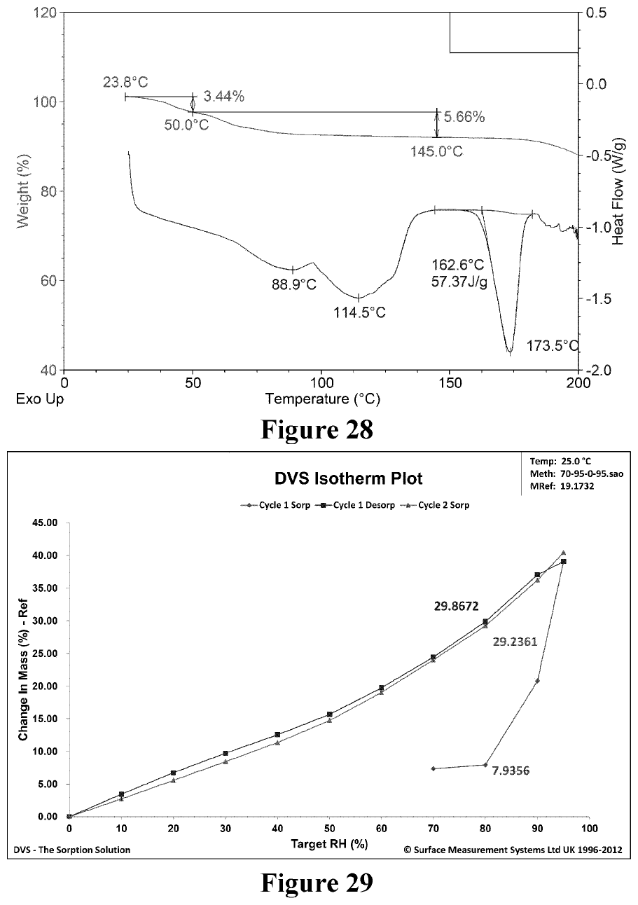

XRPD pattern of fumarate Type A was displayed in FIG. 26. ¹H NMR result of fumarate Type A (FIG. 27) showed the molar ratio of acid/freebase was 0.99. TGA/DSC curves of fumarate Type A were displayed in FIG. 28, wherein a weight loss of 9.1% up to 145° C. and endotherm peaks at 88.9° C., 114.5 (broad) and 162.6 (onset) ° C. (peak) were observed.

DVS testing on fumarate Type A was performed starting from 25° C./70% RH. As the result in FIG. 29 showed that obvious water uptake (˜21%) was observed when humidity increased, and solid was slightly sticky after DVS test.

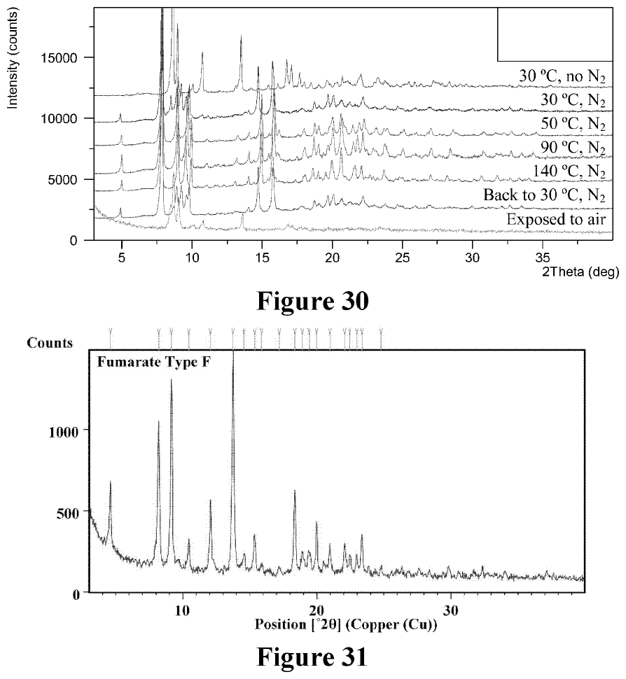

For further identification, VT-XRPD was performed on fumarate Type A. As VT-XRPD result showed in FIG. 30, form change was observed after heating fumarate Type A to 50° C., 90° C. and 140° C. with protection of N₂ (new form was assigned as fumarate Type K).

XRPD pattern of fumarate Type K was displayed in FIG. 46.

TABLE 10
X-ray Diffraction Pattern of Compound A fumarate Type A

| | Diffraction | | Relative |
|---|---|---|---|
| | angle | d-spacing | intensity |
| Peak# | (°2-theta) | [Å] | (%) |
| 1 | 8.69 | 10.18 | 100.00 |
| 2 | 9.01 | 9.82 | 12.32 |
| 3 | 10.11 | 8.75 | 3.11 |
| 4 | 10.77 | 8.21 | 17.86 |
| 5 | 13.48 | 6.57 | 5.31 |
| 6 | 16.18 | 5.48 | 2.36 |
| 7 | 16.80 | 5.28 | 11.18 |
| 8 | 17.14 | 5.17 | 9.22 |
| 9 | 17.74 | 5.00 | 5.14 |
| 10 | 18.54 | 4.79 | 1.67 |
| 11 | 19.69 | 4.51 | 3.52 |
| 12 | 22.09 | 4.02 | 2.64 |
| 13 | 23.37 | 3.81 | 2.80 |

TABLE 17
X-ray Diffraction Pattern of Compound A fumarate Type K

| | Diffraction | | Relative |
|---|---|---|---|
| | angle | d-spacing | intensity |
| Peak# | (°2-theta) | [Å] | (%) |
| 1 | 4.87 | 18.15 | 5.66 |
| 2 | 7.84 | 11.28 | 100.00 |
| 3 | 8.90 | 9.93 | 17.72 |
| 4 | 9.22 | 9.59 | 17.98 |
| 5 | 9.58 | 9.23 | 12.20 |
| 6 | 14.00 | 6.33 | 3.84 |
| 7 | 14.69 | 6.03 | 26.28 |
| 8 | 15.75 | 5.63 | 26.83 |
| 9 | 17.82 | 4.98 | 3.55 |
| 10 | 18.70 | 4.74 | 6.76 |
| 11 | 19.02 | 4.67 | 4.47 |
| 12 | 19.65 | 4.52 | 9.56 |
| 13 | 20.06 | 4.43 | 9.77 |
| 14 | 20.64 | 4.30 | 7.09 |
| 15 | 21.21 | 4.19 | 5.07 |
| 16 | 22.17 | 4.01 | 8.27 |
| 17 | 22.98 | 3.87 | 2.73 |
| 18 | 23.77 | 3.74 | 3.23 |
| 19 | 24.65 | 3.61 | 2.68 |
| 20 | 25.90 | 3.44 | 1.70 |
| 21 | 26.85 | 3.32 | 1.86 |
| 22 | 29.94 | 2.98 | 1.33 |
| 23 | 32.08 | 2.79 | 1.30 |
| 24 | 32.64 | 2.74 | 2.09 |
| 25 | 33.48 | 2.68 | 1.74 |

### Example 17. Fumarate Type D and Type L

Crystalline form of fumarate Type D was obtained via the following procedure: Fumarate (11.8 g) was dissolved in EtOH (500 mL) at r. t. The solution was concentrated under vacuum at 50° C. to remove most of EtOH until the resulting material was 22 g left. The resulting material was stayed in a quiet place overnight to give a crystalline solid. The solid was rinsed with EtOH twice and dried under vacuum at 50° C. for 4 h to give fumarate type D.

Type L was obtained via heated Type D to 140° C. under nitrogen atmosphere.

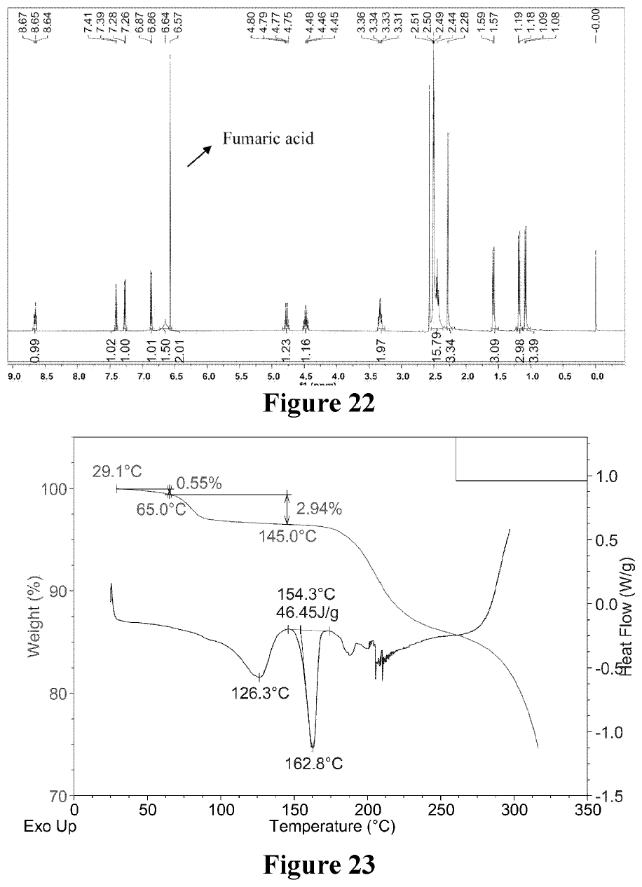

XRPD pattern of fumarate Type D was displayed in FIG. 21. ¹H NMR result of fumarate Type D (FIG. 22) showed the molar ratio of acid/freebase was 1.0. TGA/DSC curves of fumarate Type D were displayed in FIG. 23, wherein a weight loss of 3.5% up to 145° C. (which was similar to the 3.3% water content determined by KF test) and endotherm peaks at 126.3 (broad) and 154.3 (onset) ° C. (peak) were observed.

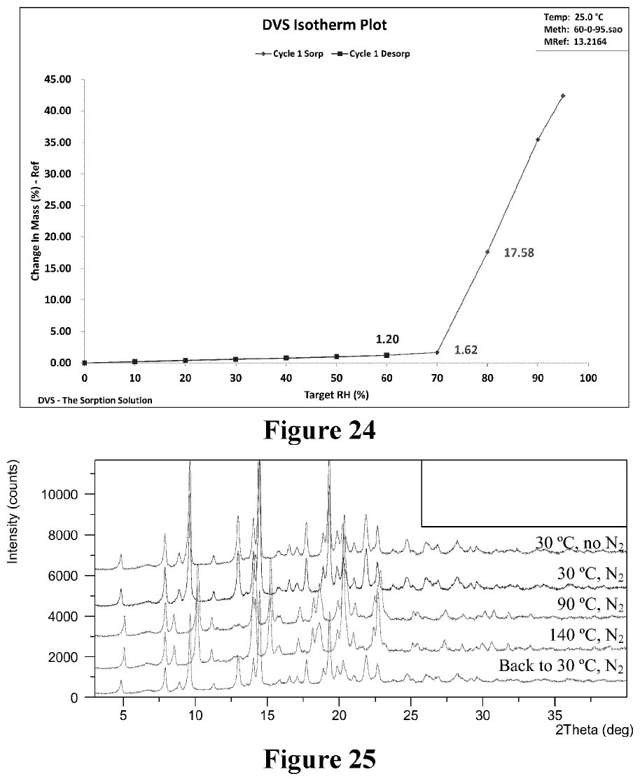

DVS test on Type D was started at 25° C./60% RH to avoid any unnecessary form change for the starting form. As shown in FIG. 24, along with humidity decreased from 60% to 0% RH and then increased from 0% to 70% RH, minor mass change (˜1.6%) was observed. Thus, fumarate Type D was speculated to be stable lower than 60% RH.

To further identify fumarate Type D, VT-XRPD was performed. As VT-XRPD result showed in FIG. 25, form change (new form was assigned as fumarate Type L) was viewed after heating fumarate Type D to 90° C. and 140° C. under nitrogen protection. After cooling back to RT, it converted back to Type D under N2 flow (relative humidity<10%).

XRPD pattern of fumarate Type L was displayed in FIG. 47.

TABLE 16
X-ray Diffraction Pattern of Compound A fumarate Type D

| | Diffraction | | Relative |
|---|---|---|---|
| | angle | d-spacing | intensity |
| Peak# | (°2-theta) | [Å] | (%) |
| 1 | 4.83 | 18.29 | 29.25 |
| 2 | 7.92 | 11.17 | 19.55 |
| 3 | 8.87 | 9.97 | 8.39 |
| 4 | 9.64 | 9.17 | 100.00 |
| 5 | 13.01 | 6.80 | 23.82 |
| 6 | 14.07 | 6.30 | 18.66 |
| 7 | 14.47 | 6.12 | 91.86 |
| 8 | 17.75 | 5.00 | 10.92 |
| 9 | 19.34 | 4.59 | 52.18 |
| 10 | 20.24 | 4.39 | 11.95 |
| 11 | 21.88 | 4.06 | 10.21 |
| 12 | 22.72 | 3.91 | 6.62 |
| 13 | 24.78 | 3.59 | 5.15 |
| 14 | 26.20 | 3.40 | 2.45 |
| 15 | 28.26 | 3.16 | 3.17 |
| 16 | 29.60 | 3.02 | 2.90 |

TABLE 17
X-ray Diffraction Pattern of Compound A fumarate Type L

| | Diffraction | | Relative |
|---|---|---|---|
| | angle | d-spacing | intensity |
| Peak# | (°2-theta) | [Å] | (%) |
| 1 | 5.05 | 17.50 | 16.01 |
| 2 | 7.89 | 11.21 | 26.39 |
| 3 | 8.51 | 10.39 | 15.30 |
| 4 | 10.11 | 8.75 | 61.62 |
| 5 | 11.11 | 7.96 | 11.52 |
| 6 | 13.98 | 6.33 | 50.51 |
| 7 | 14.14 | 6.26 | 49.52 |
| 8 | 15.16 | 5.84 | 59.80 |
| 9 | 15.77 | 5.62 | 11.36 |
| 10 | 17.15 | 5.17 | 16.82 |
| 11 | 18.15 | 4.89 | 24.67 |
| 12 | 18.43 | 4.82 | 23.24 |
| 13 | 18.60 | 4.77 | 28.23 |
| 14 | 19.86 | 4.47 | 22.34 |
| 15 | 20.27 | 4.38 | 100.00 |
| 16 | 20.96 | 4.24 | 19.20 |
| 17 | 22.36 | 3.98 | 22.13 |
| 18 | 22.69 | 3.92 | 50.04 |
| 19 | 25.11 | 3.55 | 8.58 |
| 20 | 25.43 | 3.50 | 8.52 |
| 21 | 27.32 | 3.26 | 10.58 |
| 22 | 28.54 | 3.13 | 5.79 |
| 23 | 29.93 | 2.99 | 6.07 |
| 24 | 30.60 | 2.92 | 7.27 |
| 25 | 31.73 | 2.82 | 5.14 |
| 26 | 33.26 | 2.69 | 2.94 |
| 27 | 37.74 | 2.38 | 1.94 |
| 28 | 38.76 | 2.32 | 2.27 |

### Example 18. Fumarate Type F and Type M

Crystalline form of fumarate Type F can be obtained via the following procedures: Fumarate (20.8 mg) was dissolved in EtOH (0.3 mL). To the mixture was added n-heptane (0.6 mL) dropwise. The mixture was stirred at r. t. overnight. The solid was separated by centrifugal separation.

Type M was obtained via heated Type F to 140° C. under nitrogen atmosphere, and cooled to 30° C.

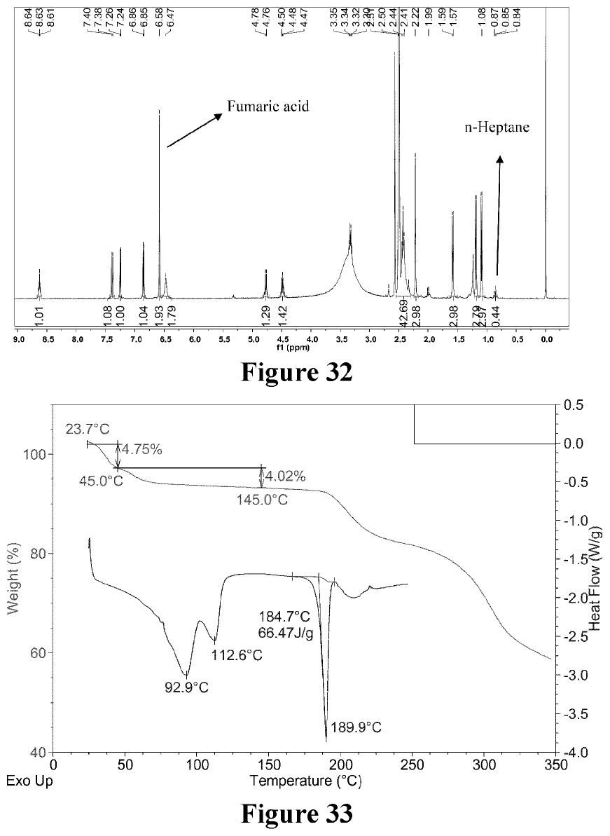

XRPD pattern of fumarate Type F was displayed in FIG. 31. ¹H NMR result of fumarate Type F (FIG. 32) showed the molar ratio of acid/freebase was 0.97. The TGA/DSC result (FIG. 33) showed a weight loss of 8.8% up to 145° C., and two broad endotherms around 92.9° C. and 112.6° C. (peak) and one sharp endotherm at 184.7° C. (onset) before decomposition.

DVS testing on fumarate Type F was started at 25° C./80% RH to avoid any unnecessary form change for the starting form. DVS result in FIG. 39 showed that obvious water uptake (˜17%) was observed when humidity increased.

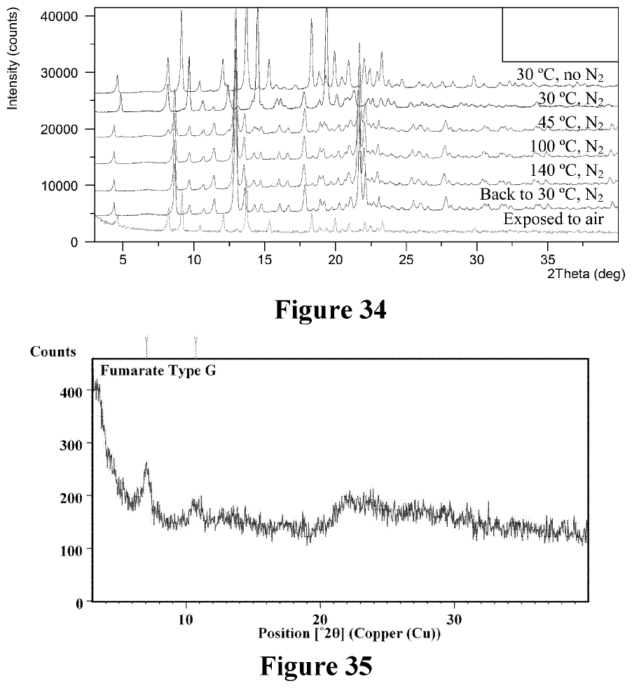

To further identify fumarate Type F, VT-XRPD was performed. As VT-XRPD result showed in FIG. 34, form change was observed after heating fumarate Type F to 100° C. and 140° C. with protection of N₂ (new form was assigned as fumarate Type M). Combined with step weight loss in TGA (8.8%) and limited solvent detected in ¹H NMR, fumarate Type F was speculated to be a hydrate. After exposed to air for 10 mins, fumarate Type M changed back to Type F, indicating that fumarate Type M was a metastable anhydrate.

XRPD pattern of fumarate Type M was displayed in FIG. 48.

TABLE 20
X-ray Diffraction Pattern of Compound A fumarate Type F

| | Diffraction | | Relative |
|---|---|---|---|
| | angle | d-spacing | intensity |
| Peak# | (°2-theta) | [Å] | (%) |
| 1 | 4.60 | 19.22 | 30.02 |
| 2 | 8.20 | 10.79 | 62.37 |
| 3 | 9.16 | 9.66 | 86.23 |
| 4 | 10.44 | 8.47 | 14.11 |
| 5 | 12.06 | 7.34 | 32.53 |
| 6 | 13.74 | 6.45 | 100.00 |
| 7 | 14.55 | 6.09 | 7.97 |
| 8 | 15.33 | 5.78 | 17.78 |
| 9 | 15.86 | 5.59 | 4.40 |
| 10 | 17.19 | 5.16 | 3.11 |
| 11 | 18.33 | 4.84 | 39.14 |
| 12 | 18.90 | 4.70 | 9.69 |
| 13 | 19.42 | 4.57 | 10.80 |
| 14 | 19.97 | 4.45 | 24.31 |
| 15 | 20.96 | 4.24 | 14.56 |
| 16 | 22.06 | 4.03 | 14.77 |
| 17 | 22.45 | 3.96 | 9.13 |
| 18 | 22.96 | 3.87 | 9.76 |
| 19 | 23.33 | 3.81 | 18.01 |
| 20 | 24.78 | 3.59 | 4.23 |

TABLE 18
X-ray Diffraction Pattern of Compound A fumarate Type M

| | Diffraction | | Relative |
|---|---|---|---|
| | angle | d-spacing | intensity |
| Peak# | (°2-theta) | [Å] | (%) |
| 1 | 4.35 | 20.33 | 8.44 |
| 2 | 8.65 | 10.23 | 33.84 |
| 3 | 9.68 | 9.14 | 6.71 |
| 4 | 10.69 | 8.27 | 4.58 |
| 5 | 11.44 | 7.74 | 10.71 |
| 6 | 12.96 | 6.83 | 100.00 |
| 7 | 13.58 | 6.52 | 18.29 |
| 8 | 14.28 | 6.20 | 6.87 |
| 9 | 14.76 | 6.00 | 7.75 |
| 10 | 15.52 | 5.71 | 2.97 |
| 11 | 16.04 | 5.52 | 8.02 |
| 12 | 16.67 | 5.32 | 6.65 |
| 13 | 17.83 | 4.98 | 20.44 |
| 14 | 18.41 | 4.82 | 4.39 |
| 15 | 18.92 | 4.69 | 10.65 |
| 16 | 19.18 | 4.63 | 9.41 |
| 17 | 19.73 | 4.50 | 5.46 |
| 18 | 20.25 | 4.38 | 8.16 |
| 19 | 20.74 | 4.28 | 7.34 |
| 20 | 21.04 | 4.22 | 11.88 |
| 21 | 21.68 | 4.10 | 67.60 |
| 22 | 22.09 | 4.02 | 28.42 |
| 23 | 22.38 | 3.97 | 8.30 |
| 24 | 22.65 | 3.93 | 9.06 |
| 25 | 23.07 | 3.86 | 12.65 |
| 26 | 23.41 | 3.80 | 6.29 |
| 27 | 24.00 | 3.71 | 6.22 |
| 28 | 24.69 | 3.61 | 5.06 |
| 29 | 25.52 | 3.49 | 9.91 |
| 30 | 26.01 | 3.43 | 8.10 |
| 31 | 26.53 | 3.36 | 6.01 |
| 32 | 27.81 | 3.21 | 12.73 |
| 33 | 28.16 | 3.17 | 6.62 |
| 34 | 28.76 | 3.10 | 5.81 |
| 35 | 29.28 | 3.05 | 5.20 |
| 36 | 29.77 | 3.00 | 5.02 |
| 37 | 30.55 | 2.93 | 7.85 |
| 38 | 30.79 | 2.90 | 7.36 |
| 39 | 31.74 | 2.82 | 7.33 |
| 40 | 31.99 | 2.80 | 7.29 |
| 41 | 32.39 | 2.76 | 6.23 |
| 42 | 33.46 | 2.68 | 5.25 |
| 43 | 34.16 | 2.62 | 7.46 |
| 44 | 34.43 | 2.61 | 7.17 |
| 45 | 35.00 | 2.56 | 7.71 |
| 46 | 35.77 | 2.51 | 5.04 |
| 47 | 36.34 | 2.47 | 5.18 |
| 48 | 36.81 | 2.44 | 4.74 |
| 49 | 37.86 | 2.38 | 5.85 |
| 50 | 38.56 | 2.33 | 5.55 |
| 51 | 39.04 | 2.31 | 5.27 |
| 52 | 39.55 | 2.28 | 9.10 |

### Example 19. Fumarate Type G

Crystalline form of fumarate Type G can be obtained via solid vapor diffusion in H₂O for 8 days, followed by air-drying at RT overnight. A 3 mL of bottle contented Fumarate (19.5 mg) was placed in a 20 mL of bottle contented water (4 mL) for 8 days. The solid was collected.

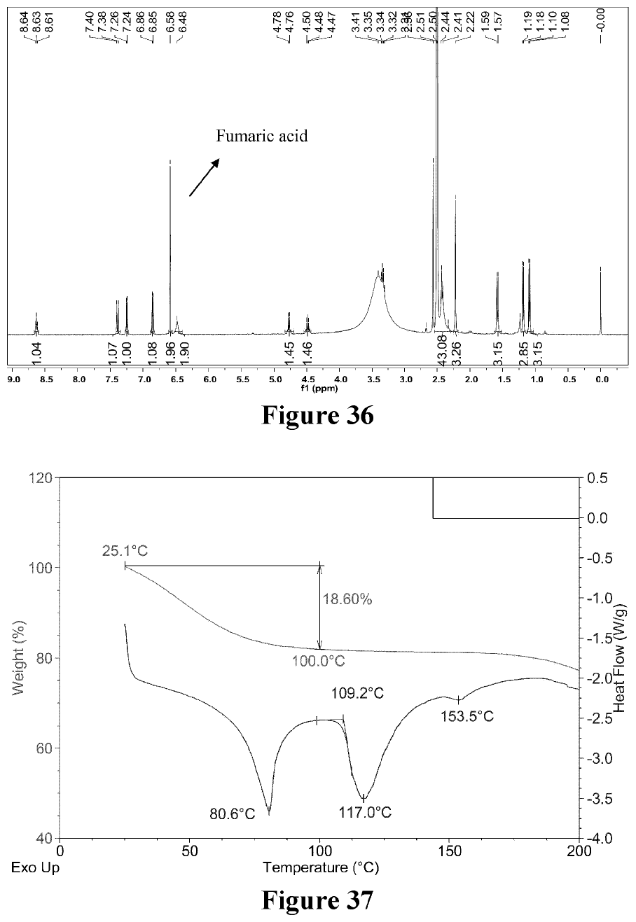

XRPD pattern of fumarate Type G was displayed in FIG. 35. ¹H NMR result of fumarate Type G (FIG. 36) showed the molar ratio of acid/freebase was 0.98. The TGA/DSC result (FIG. 37) showed a weight loss of 18.6% up to 100° C., and three endotherms around 80.6° C., 117.0° C. and 153.5° C. (peak) before decomposition.

TABLE 21
X-ray Diffraction Pattern of Compound A fumarate Type G

| | Diffraction | | Relative |
|---|---|---|---|
| | angle | d-spacing | intensity |
| Peak# | (°2-theta) | [Å] | (%) |
| 1 | 7.06 | 12.52 | 100.00 |
| 2 | 10.71 | 8.26 | 35.23 |

### Example 20. Fumarate Type H

Crystalline form of fumarate Type H was obtained via the following procedures: Fumarate (59.5 mg) was dissolved in a mixture of 1,4-dioxane and water (9/1, v/v, 0.5 mL). The mixture was stirred at r. t. for 2 days and at −4° C. for 8 days. The solid was collected by filtration.

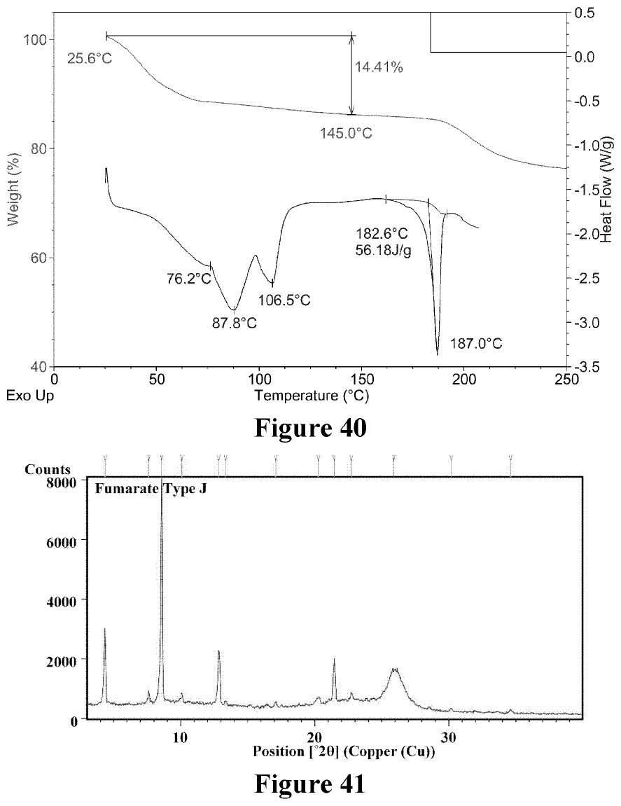

XRPD pattern of fumarate Type H was displayed in FIG. 38. ¹H NMR result of fumarate Type H (FIG. 39) showed the molar ratio of acid/freebase was 1.0. The TGA/DSC result (FIG. 40) showed a weight loss of 14.4% up to 145° C., and multiple endotherms around 76.2° C., 87.8° C., 106.5° C. (peak) and 182.6° C. (onset) before decomposition.

TABLE 22
X-ray Diffraction Pattern of Compound A fumarate Type H

| | Diffraction | | Relative |
|---|---|---|---|
| | angle | d-spacing | intensity |
| Peak# | (°2-theta) | [Å] | (%) |
| 1 | 8.13 | 10.87 | 97.60 |
| 2 | 8.43 | 10.49 | 72.36 |
| 3 | 9.37 | 9.44 | 100.00 |
| 4 | 11.71 | 7.56 | 31.76 |
| 5 | 12.21 | 7.25 | 25.10 |
| 6 | 12.92 | 6.85 | 39.79 |
| 7 | 15.69 | 5.65 | 16.59 |
| 8 | 20.13 | 4.41 | 38.10 |
| 9 | 22.15 | 4.01 | 45.37 |
| 10 | 23.20 | 3.83 | 31.73 |

### Example 21. Fumarate Type J

Crystalline form of fumarate Type J can be obtained via recrystallization of fumarate Type D in EtOH. Fumarate (500.5 mg) was dissolved in EtOH (3.17 mL) at 70° C. The resulting clear solution was stirred at r. t. for 2 days. The solid was collected by Centrifugal separation. XRPD pattern of fumarate Type J was displayed in FIG. 41.

TABLE 17
X-ray Diffraction Pattern of Compound A fumarate Type J

| | Diffraction | | Relative |
|---|---|---|---|
| | angle | d-spacing | intensity |
| Peak# | (°2-theta) | [Å] | (%) |
| 1 | 4.35 | 20.34 | 33.73 |
| 2 | 7.61 | 11.62 | 6.16 |
| 3 | 8.58 | 10.31 | 100.00 |
| 4 | 10.08 | 8.78 | 5.50 |
| 5 | 12.84 | 6.90 | 24.22 |
| 6 | 13.33 | 6.64 | 1.99 |
| 7 | 17.08 | 5.19 | 2.12 |
| 8 | 20.26 | 4.38 | 2.87 |
| 9 | 21.44 | 4.14 | 19.19 |
| 10 | 22.73 | 3.91 | 3.72 |
| 11 | 25.91 | 3.44 | 12.62 |
| 12 | 30.18 | 2.96 | 1.39 |
| 13 | 34.60 | 2.59 | 1.53 |

### Example 22. Fumarate Type E

Crystalline form of fumarate Type E was obtained via the following procedure: fumarate (20.7 mg) was dissolved in NMP (0.2 mL). To the clear solution was added EtOAc (1.8 mL) dropwise. The resulting mixture was stirred at room temperature over night.

As displayed by XRPD pattern in FIG. 42, fumarate Type E was observed from the wet sample obtained by anti-solvent addition in NMP/EtOAc, and it transformed to Type A after air-drying overnight.

TABLE 17
X-ray Diffraction Pattern of Compound A fumarate Type E

| | Diffraction | | Relative |
|---|---|---|---|
| | angle | d-spacing | intensity |
| Peak# | (°2-theta) | [Å] | (%) |
| 1 | 7.56 | 11.69 | 16.90 |
| 2 | 8.93 | 9.90 | 46.71 |
| 3 | 9.30 | 9.51 | 21.25 |
| 4 | 10.73 | 8.24 | 6.05 |
| 5 | 11.36 | 7.79 | 8.09 |
| 6 | 12.00 | 7.38 | 2.91 |
| 7 | 13.48 | 6.57 | 40.81 |
| 8 | 13.99 | 6.33 | 100.00 |
| 9 | 14.50 | 6.11 | 28.38 |
| 10 | 15.93 | 5.56 | 11.61 |
| 11 | 17.95 | 4.94 | 8.15 |
| 12 | 18.70 | 4.75 | 30.89 |
| 13 | 19.00 | 4.67 | 12.06 |
| 14 | 20.22 | 4.39 | 8.36 |
| 15 | 20.70 | 4.29 | 23.83 |
| 16 | 21.28 | 4.18 | 6.55 |
| 17 | 21.87 | 4.06 | 11.06 |
| 18 | 22.78 | 3.90 | 6.46 |
| 19 | 23.73 | 3.75 | 11.65 |
| 20 | 24.20 | 3.68 | 7.68 |
| 21 | 25.60 | 3.48 | 6.98 |
| 22 | 26.29 | 3.39 | 7.17 |
| 23 | 26.81 | 3.33 | 5.81 |
| 24 | 28.21 | 3.16 | 6.25 |
| 25 | 28.48 | 3.13 | 7.21 |

### Example 23. Fumarate Type I

Crystalline form of fumarate Type I can be obtained via solid vapor diffusion in EtOH for 8 days, followed by air-drying at RT overnight. A 3 mL of bottle contented Fumarate (20 mg) was placed in a 20 mL of bottle contented EtOH (4 mL) for 8 days. The solid was collected.

As displayed by XRPD pattern in FIG. 43.

TABLE 23
X-ray Diffraction Pattern of Compound A fumarate Type I

| | Diffraction | | Relative |
|---|---|---|---|
| | angle | d-spacing | intensity |
| Peak# | (°2-theta) | [Å] | (%) |
| 1 | 8.74 | 10.12 | 100.00 |
| 2 | 9.35 | 9.46 | 56.49 |
| 3 | 10.80 | 8.19 | 10.95 |
| 4 | 13.13 | 6.75 | 61.55 |
| 5 | 13.99 | 6.33 | 43.51 |

### Example 24: Solid-State Stability Tests

Fumarate Type D and Type F were further evaluated by solid-state stability tests under 25° C./60% RH and 40° C./75% RH for one week.

In the experiments, about 15 mg of solids was added into an HPLC vial, which was then sealed with parafilm and pricked with 10 holes. Place the vial under corresponding condition and test the solids by HPLC and XRPD after one week. The results were summarized in Table 24 below.

TABLE 24
Summary of solid-state stability evaluation

| | | Initial | | One week | |
|---|---|---|---|---|---|
| | | | HPLC | | HPLC |
| Solid form | Condition | XRPD | (area %) | XRPD | (area %) |
| Fumarate | 25° C./60% RH | Fumarate | 99.9 | Fumarate | 99.9 |
| Type D | 40° C./75% RH | Type D | | Type D | |
| Fumarate | 25° C./60% RH | Fumarate | 99.9 | Fumarate | 99.9 |
| Type F | 40° C./75% RH | Type F | | Type F | |

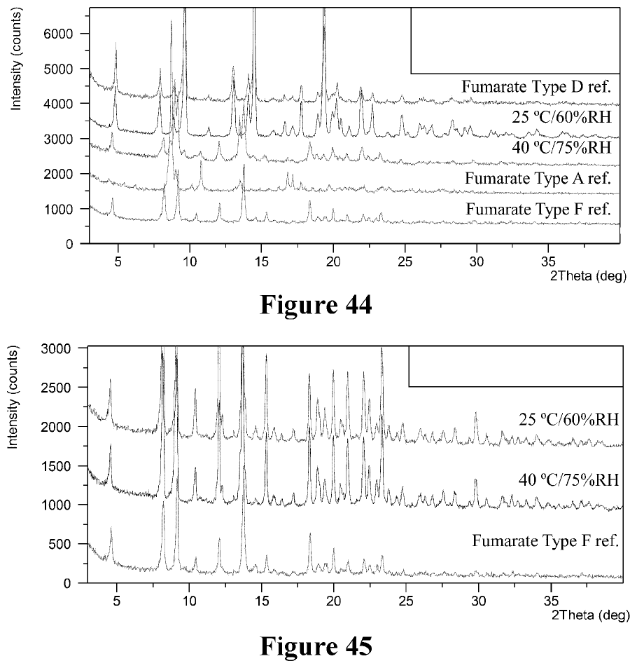

For fumarate Type D: XRPD results in FIG. 44 showed that no form change was observed after storage at 25° C./60% RH and 40° C./75% RH for one week. HPLC results in Table 25 showed that no obvious difference of HPLC purity was observed after storage under test conditions.

TABLE 25
HPLC purity of fumarate Type D

| | Area (%) | | |
|---|---|---|---|
| | | 25° C./60% RH | 40° C./75% RH |
| RRT | Initial | for 1 week | for 1 week |
| 1.00 | 99.92 | 99.92 | 99.90 |
| 1.08 | 0.03 | 0.02 | 0.03 |
| 1.13 | 0.05 | 0.06 | 0.07 |

For fumarate Type F: XRPD results in FIG. 45 showed that no form change was observed after storage at 25° C./60% RH or 40° C./75% RH for one week. HPLC results in Table 26 showed that no obvious difference of HPLC purity was observed after storage at 25° C./60% RH or 40° C./75% RH for one week.

TABLE 26
HPLC purity of fumarate Type F

| | | Area (%) | |
|---|---|---|---|
| | | 25° C./60% RH | 40° C./75% RH |
| RRT | Initial | for 1 week | for 1 week |
| 1.00 | 99.95 | 99.88 | 99.91 |
| 1.06 | NA | 0.05 | NA |
| 1.08 | NA | 0.02 | 0.03 |
| 1.13 | 0.05 | 0.05 | 0.06 |

### Example 25: Pharmacokinetic Properties of Different Salts in Sprague-Dawley Rats After Oral Administrations (PO)

### Dose Formulation Preparation

The oral dosing solution was prepared as follows: 5.0 mg of a test compound was weighed and dispersed in 10 mL of 0.5% methyl cellulose (MC). The final concentration of the test compound is 1 mg·mL⁻¹ (Calculated by free freebase).

### Animals

Male Sprague-Dawley rats (also summarized in Table 27) were housed in solid bottom polypropylene cages with sterilized bedding and receive sterilized diet and sterilized water. The room was controlled and monitored for humidity (targeted mean range 40% to 70%) and temperature (targeted mean range 18° C. to 26° C.) with 10 to 20 air changes/hour. The light cycle was maintained at 12-h light and 12-h dark. Only animals that appeared to be healthy were selected for this study based on overall health, body weight, or other relevant information. The animals were treated in accordance with a certain treatment schedule as summarized in Table 28.

TABLE 27
Animal Information

| | | | | | Weight | | |
|---|---|---|---|---|---|---|---|
| Genus | Gender | Species | Source | Age | (g) | Reserved | Selected |
| Rat | Male | Sprague | Vital | 8 | 220-250 | 7 | 6 |
| | | Dawley | River | weeks | | | |
| | | (SD) | | | | | |

TABLE 28
Animal Treatment Schedule

| | Dose | | | | | | |
|---|---|---|---|---|---|---|---|
| | Level | Concentration. | | Dosing | | | Sampling |
| Quantity | (mg · kg⁻¹) | (mg · mL⁻¹) | Vehicle | Route | Regimen | Fasted/Fed | Time |
| 3/Group | 10.0 | 1.0 | 0.5% | Oral | Single | Fasted | Pre-dose, |
| | | | MC | | | | 15, 30 min, |
| | | | | | | | 1, 2, 4, 8, 24 |
| | | | | | | | h |

### Study Design

All procedures performed on animals were in accordance with established guidelines and reviewed and approved by an independent institutional review board.

The male Sprague-Dawley rats were fasted overnight with free access to drinking water prior to treatment. On day 1, the animals were weighed and actual dose volume for each animal was calculated using the formula below:

Dose Volume (mL)=[Nominal Dose (mg·kg⁻¹)/Dose Concentration (mg·mL⁻¹)]×Animal Body Weight (kg)

Three rats for each group were given a single oral dose of 10 mg·kg⁻¹. The dosing solutions were freshly prepared prior to dose administration. The actual body weights and actual volume injected were recorded accordingly. Four hours after dosing, the rats were allowed to intake food.

Blood samples (˜150 μL) were collected at different times from the jugular vein catheter into EDTA-K₂ coated tubes. Whole blood was processed by centrifugation at 3000 g for 10 min. Plasma samples were collected and kept at −80° C. freezer prior to analysis. The blood sampling time was recorded accordingly.

### Sample Test

The dose samples of PO were diluted with MeOH:H₂O (4:1, v/v) to achieve the concentration of 2 μg·mL⁻¹, respectively. Then, 2.5 μL of the diluted samples were added with 47.5 μL blank plasma, and then were handled as the plasma sample procedure. An aliquot of 10 μL of the mixture was injected into the LC-MS/MS system. The pharmacokinetic (PK) data of the test compounds were generated as shown in Table 29.

TABLE 29
Pharmacokinetic properties of D-Tartarate and Fumarate

| | | D-Tartarate | Fumarate |
|---|---|---|---|
| | | (acid/base = | (acid/base = 1:1, |
| PK Parameters | | 1.5:1) | Type D) |
| Dose (calculated | mg · kg⁻¹ | 10 | 10 |
| as free base) | | | |
| Kₑₗ | h⁻¹ | 0.06 | 0.0687 |
| t_{1/2} | h | 12 | 11 |
| tₘₐₓ | h | 2.7 | 2.45 |
| Cₘₐₓ | ng · mL⁻¹ | 14.8 | 48.3 |
| AUC₀₋ₜ | h · ng · mL⁻¹ | 54.9 | 210 |
| AUC_{0-inf} | h · ng · mL⁻¹ | 147.7 | 356 |

| | | |
|---|---|---|
| F % (n = 3) | 3.4 | 13.6 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

1. A pharmaceutically acceptable salt of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide, wherein said pharmaceutically acceptable salts are inorganic salt(s) or organic salt(s).

2. The salt according to claim 1, which is in a solid state.

3. The salt according to claim 1, wherein:
(a) the salt is an inorganic salt selected from the group consisting of hydrochloride, sulphate, phosphate, hydrobromide and/or nitrate; or is organic salt selected from fumarate, tartrate (L-tartrate or D-tartrate), laurate, stearate, gentisate, nicotinate, aspartate, succinate, adipate, malate (L-malate), citrate, glycolate, gluconate (D-gluconate), lactate (DL-lactate), acetate, benzene sulfonate, methanesulfonate, mesylate, benzoate, naphthalene sulfonate, oxalate, and any combination thereof; or
(b) the salt is selected from the group consisting of fumarate, L-tartrate, D-tartrate, sulphate, tartrate, laurate, stearate, gentisate, and nicotinate.

4. (canceled)

5. The salt according to claim 3, wherein the salt is fumarate.

6. The salt according to claim 5, wherein the salt is a compound of Formula (I): 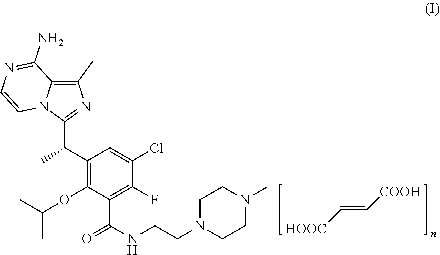
wherein n is a number from about 0.5 to about 2.0.

7. The salt according to claim 6, wherein n is a number:
(a) from about 0.5 to about 1.5;
(b) selected from the group consisting of 0.5±0.1, 1.0±0.2 and 1.5±0.2;
(c) selected from 1.0±0.1, 1.1±0.1 and 1.5±0.1;
(d) selected from about 0.95 to about 1.05, about 1.05 to about 1.15, or about 1.45 to about 1.55;
(e) selected from about 0.98 to about 1.02, about 1.08 to about 1.12 or about 1.48 to about 1.52; or
(f) selected from 1.0, 1.1 and 1.5.

8. (canceled)

9. The salt according to claim 3, wherein:
(a) the salt is tartrate;
(b) the salt is D-tartrate; and/or
(c) the salt is a compound of Formula (II): 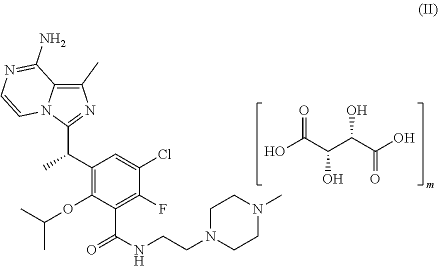
wherein m is a number from about 0.5 to about 2.0.

10. (canceled)

11. The salt according to claim 9, wherein:
(a) m is a number selected from about 0.5 to about 1.5;
(b) m is a number selected from the group consisting of 0.5±0.1, 1.0±0.2 and 1.5±0.2;
(c) m is a number selected from 1.0±0.1 and 1.5±0.1;
(d) m is 0.95˜1.05 or 1.45˜1.55;
(e) m is 0.98˜1.02 or 1.48˜1.52;
(f) m is 1.0 or 1.5.

12. (canceled)

13. A pharmaceutical composition comprising a therapeutically effective amount of the salts according to claim 1, and optionally one or more pharmaceutically acceptable carrier(s).

14. A method for treating or preventing a disorder or a disease selected from the group consisting of inflammatory disorder, autoimmune disease, and a cancer, comprising administering to a subject in need thereof a therapeutically effective amount of the pharmaceutical composition of claim 13.

15. A process for preparing the salts of claim 1, comprising:
(a) mixing a free base of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide and corresponding acid in an appropriate solvent to form a suspension;
(b) isolating a solid from the suspension to obtain the salt of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide; and optionally
(c) drying the solid in vacuum.

16. The process according to claim 15, wherein:
(a) the corresponding acid is selected from the group consisting of hydrochloric acid, sulfuric acid, phosphoric acid, hydrobromic acid, nitric acid, fumaric acid, L-tartaric acid, D-tartaric acid, lauric acid, stearic acid, gentistic acid, nicotinic acid, aspartic acid, succinic acid, adipic acid, malic acid(L-malic acid), citric acid, ascobic acid (L-ascobic acid), glycolic acid, gluconic acid (D-gluconic acid), lactic acid (DL-lactic acid), acetic acid, benzene sulfonic acid, methanesulfonic acid, benzoic acid, naphthalene sulfonic acid, oxalic acid, and any combination thereof;
(b) the corresponding acid is selected from the group consisting of sulfuric acid, fumaric acid, L-tartaric acid, D-tartaric acid, lauric acid, stearic acid, gentistic acid and nicotinic acid; and/or
(c) the solvent is selected from the group consisting of acetone, heptane (n-heptane), isopropyl alcohol, isopropyl acetate, 1,4-dioxane, and any combination thereof.

17. (canceled)

18. (canceled)

19. (canceled)

20. A crystalline form of a salt of Formula III 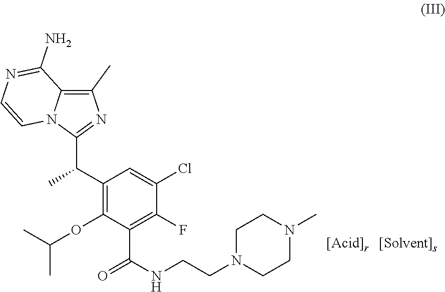 [SMILES: Cc1nc([C@@H](C)c2cc(Cl)c(F)c(C(=O)NCCN3CCN(C)CC3)c2OC(C)C)n2ccnc(N)c12]
wherein:
(a) the acid is selected from the group consisting of organic acids and inorganic acids;
(b) the solvent is selected from H₂O or organic solvents;
(c) r is a number from about 0.0 to about 5.0; and
(d) s is a number from about 0.0 to about 5.0.

21. The crystalline form of claim 20, wherein:
(a) the acid is selected from the group consisting of inorganic acid selected from Hydrochloric acid, sulfuric acid, phosphoric acid, hydrobromic acid and/or nitric acid; or organic acid selected from fumaric acid, tartaric acid (L-tartaric acid or d-tartaric acid), lauric acid, stearic acid, gentian acid, nicotinic acid, aspartic acid, succinic acid, adipic acid, malic acid (L-malic acid), citric acid, glycolic acid, gluconic acid (d-Gluconic acid), lactic acid (DL lactic acid), acetic acid, benzenesulfonic acid, methanesulfonic acid, methanesulfonic acid, benzoic acid, naphthalenesulfonic acid and oxalic acid;
(b) the acid is selected from sulfuric acid, fumaric acid, tartaric acid, L-tartaric acid, d-tartaric acid, sulfuric acid, lauric acid, stearic acid, gentian acid, and nicotinic acid;
(c) the acid is fumaric acid; and/or
(d) the solvent is selected from the group consisting of MeOH, EtOH, i-PrOH, n-PrOH, n-BuOH, t-BuOH, acetone, butanone, pentanone, H₂O, MeCN, THF, ether, propyl ether, n-heptane, hexane, 1,4-dioxane, and EtOAc.

22. The crystalline form of claim 20, wherein:
(a) r is a number from about 0.0 to about 3.0;
(b) r is a number from about 0.0 to about 2.0;
(c) r is a number selected from the group consisting of about 0.5±0.1, about 1.0±0.2 and about 1.5±0.2;
(d) r is a number selected from the group consisting of about 0.95 to about 1.05, about 1.05˜1.15 and about 1.45 to about 1.55;
(e) r is a number selected from the group consisting of about 0.98 to about 1.02, about 1.08 to about 1.12 and about 1.48˜1.52;
(f) r is 1.0, 1.1 or 1.5;
(g) s is a number selected from the group consisting of from about 0.0 to about 3.0;
(h) s is a number selected from about 0.0 to about 2.0;
(i) s is a number selected from the group consisting of 0.1±0.1, 0.5±0.1, 1.0±0.2, 1.5±0.2 and 2.0±0.2;
(j) s is a number selected from the group consisting of about 0 to about 0.2, about 0.95 to about 1.05, about 1.05 to about 1.15, about 1.45 to about 1.55, about 1.90 to about 2.10;
(k) s is about 0.98 to about 1.02, about 1.08 to about 1.12, about 1.48 to about 1.52, or about 1.95 to about 2.15; and/or
(l) s is 0, 0.1, 0.2, 1.0, 1.1, 1.5 or 2.0.

23. (canceled)

24. (canceled)

25. A crystalline form of claim 20, wherein the crystalline form is:

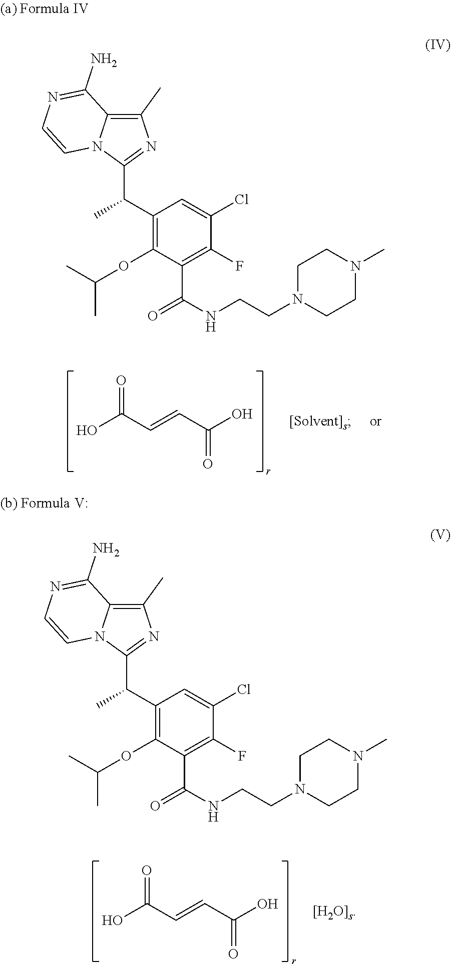

26. (canceled)

27. A crystalline form of claim 25(b), wherein:
(a) r is a number from about 0.0 to about 3.0;
(b) r is a number from about 0.0 to about 2.0;
(c) r is a number selected from the group consisting of 0.5±0.1, 1.0±0.2 and 1.5±0.2;
(d) r is about 0.95 to about 1.05, about 1.05 to about 1.15 or about 1.45 to about 1.55;
(e) r is about 0.98 to about 1.02, about 1.08 to about 1.12 or about 1.48 to about 1.52;
(f) r is 1.0, 1.1 or 1.5;
(g) s is a number from about 0.0 to about 3.0;
(h) s is a number from about 0.0 to about 2.0;
(i) s is a number selected from the group consisting of 0.1±0.1, 0.5±0.1, 1.0±0.2 and 1.5±0.2;
(j) s is about 0 to about 0.2, about 0.95 to about 1.05, about 1.05 to about 1.15 or about 1.45 to about 1.55;
(k) s is about 0.98 to about 1.02, about 1.08 to about 1.12 or about 1.48 to about 1.52;
(l) s is 0, 0.1, 0.2, 1.0, 1.1 or 1.5;
(m) s is 0.

28. A crystalline form of claim 20, which is selected from the group consisting of:
fumarate Crystalline Form A, characterized by a powder X-ray diffraction pattern comprising three, four, five, six, seven, eight, nine or more diffraction peaks having 2θ angle values independently selected from the group consisting of 8.69±0.2, 9.01±0.2, 10.11±0.2, 10.77±0.2, 13.48±0.2, 16.18±0.2, 16.80±0.2, 17.14±0.2, 17.74±0.2, 18.54±0.2, 19.69±0.2, 22.09±0.2, and 23.37±0.2;
fumarate Crystalline Form D, characterized by a powder X-ray diffraction pattern comprising three, four, five, six, seven, eight, nine or more diffraction peaks having 2θ angle values independently selected from the group consisting of 4.83±0.2, 7.92±0.2, 8.87±0.2, 9.64±0.2, 13.01±0.2, 14.07±0.2, 14.47±0.2, 17.75±0.2, 19.34±0.2, 20.24±0.2, 21.88±0.2, 22.72±0.2, 24.78±0.2, 26.20±0.2, 28.26±0.2, and 29.60±0.2;
fumarate Crystalline Form E, characterized by a powder X-ray diffraction pattern comprising three, four, five, six, seven, eight, nine or more diffraction peaks having 2θ angle values independently selected from the group consisting of 7.56±0.2, 8.93±0.2, 9.30±0.2, 10.73±0.2, 11.36±0.2, 12.00±0.2, 13.48±0.2, 13.99±0.2, 14.50±0.2, 15.93±0.2, 17.95±0.2, 18.70±0.2, 19.00±0.2, 20.22±0.2, 20.70±0.2, 21.28±0.2, 21.87±0.2, 22.78±0.2, 23.73±0.2, 24.20±0.2, 25.60±0.2, 26.29±0.2, 26.81±0.2, 28.21±0.2, and 28.48±0.2;
fumarate Crystalline Form F, characterized by a powder X-ray diffraction pattern comprising three, four, five, six, seven, eight, nine or more diffraction peaks having 2θ angle values independently selected from the group consisting of 4.60±0.2, 8.20±0.2, 9.16±0.2, 10.44±0.2, 12.06±0.2, 13.74±0.2, 14.55±0.2, 15.33±0.2, 15.86±0.2, 17.19±0.2, 18.33±0.2, 18.90±0.2, 19.42±0.2, 19.97±0.2, 20.96±0.2, 22.06±0.2, 22.45±0.2, 22.96±0.2, 23.33±0.2, and 24.78±0.2;
fumarate Crystalline Form G, characterized by a powder X-ray diffraction pattern comprising three, four, five, six, seven, eight, nine or more diffraction peaks having 2θ angle values independently selected from the group consisting of 7.06±0.2, and 10.71±0.2;
fumarate Crystalline Form H, characterized by a powder X-ray diffraction pattern comprising three, four, five, six, seven, eight, nine or more diffraction peaks having 2θ angle values independently selected from the group consisting of 8.13±0.2, 8.43±0.2, 9.37±0.2, 11.71±0.2, 12.21±0.2, 12.92±0.2, 15.69±0.2, 20.13±0.2, 22.15±0.2[[,]] and 23.20±0.2;
fumarate Crystalline Form I, characterized by a powder X-ray diffraction pattern comprising three, four, five, six, seven, eight, nine or more diffraction peaks having 2θ angle values independently selected from the group consisting of 8.74±0.2, 9.35±0.2, 10.80±0.2, 13.13±0.2, and 13.99±0.2;
fumarate Crystalline Form J, characterized by a powder X-ray diffraction pattern comprising three, four, five, six, seven, eight, nine or more diffraction peaks having 2θ angle values independently selected from the group consisting of 4.35±0.2, 7.61±0.2, 8.58±0.2, 10.08±0.2, 12.84±0.2, 13.33±0.2, 17.08±0.2, 20.26±0.2, 21.44±0.2, 22.73±0.2, 25.91±0.2, 30.18±0.2, and 34.60±0.2;
fumarate Crystalline Form K, characterized by a powder X-ray diffraction pattern comprising three, four, five, six, seven, eight, nine or more diffraction peaks having 2θ angle values independently selected from the group consisting of 4.87±0.2, 7.84±0.2, 8.90±0.2, 9.22±0.2, 9.58±0.2, 14.00±0.2, 14.69±0.2, 15.75±0.2, 17.82±0.2, 18.70±0.2, 19.02±0.2, 19.65±0.2, 20.06±0.2, 20.64±0.2, 21.21±0.2, 22.17±0.2, 22.98±0.2, 23.77±0.2, 24.65±0.2, 25.90±0.2, 26.85±0.2, 29.94±0.2, 32.08±0.2, 32.64±0.2, and 33.48±0.2;
fumarate Crystalline Form L, characterized by a powder X-ray diffraction pattern comprising three, four, five, six, seven, eight, nine or more diffraction peaks having 2θ angle values independently selected from the group consisting of 5.05±0.2, 7.89±0.2, 8.51±0.2, 10.11±0.2, 11.11±0.2, 13.98±0.2, 14.14±0.2, 15.16±0.2, 15.77±0.2, 17.15±0.2, 18.15±0.2, 18.43±0.2, 18.60±0.2, 19.86±0.2, 20.27±0.2, 20.96±0.2, 22.36±0.2, 22.69±0.2, 25.11±0.2, 25.43±0.2, 27.32±0.2, 28.54±0.2, 29.93±0.2, 30.60±0.2, 31.73±0.2, 33.26±0.2, 37.74±0.2, and 38.76±0.2; and
fumarate Crystalline Form M, characterized by a powder X-ray diffraction pattern comprising three, four, five, six, seven, eight, nine or more diffraction peaks having 2θ angle values independently selected from the group consisting of 4.35±0.2, 8.65±0.2, 9.68±0.2, 10.69±0.2, 11.44±0.2, 12.96±0.2, 13.58±0.2, 14.28±0.2, 14.76±0.2, 15.52±0.2, 16.04±0.2, 16.67±0.2, 17.83±0.2, 18.41±0.2, 18.92±0.2, 19.18±0.2, 19.73±0.2, 20.25±0.2, 20.74±0.2, 21.04±0.2, 21.68±0.2, 22.09±0.2, 22.38±0.2, 22.65±0.2, 23.07±0.2, 23.41±0.2, 24.00±0.2, 24.69±0.2, 25.52±0.2, 26.01±0.2, 26.53±0.2, 27.81±0.2, 28.16±0.2, 28.76±0.2, 29.28±0.2, 29.77±0.2, 30.55±0.2, 30.79±0.2, 31.74±0.2, 31.99±0.2, 32.39±0.2, 33.46±0.2, 34.16±0.2, 34.43±0.2, 35.00±0.2, 35.77±0.2, 36.34±0.2, 36.81±0.2, 37.86±0.2, 38.56±0.2, 39.04±0.2, and 39.55±0.2.

29. A crystalline form of claim 20, which is selected from the group consisting of:
fumarate salt Type A, characterized by a powder X-ray diffraction pattern comprising diffraction peaks having 2θ angle values of 8.69±0.2, 9.01±0.2 and 10.77±0.2; preferably having 2θ angle values of 8.69±0.2, 9.01±0.2, 10.77±0.2, 16.8±0.2 and 17.14±0.2; more preferably having 2θ angle values of 8.69±0.2, 9.01±0.2, 10.77±0.2, 13.48±0.2, 16.8±0.2, 17.14±0.2 and 17.74±0.2; even more preferably having 2θ angle values of 8.69±0.2, 9.01±0.2, 10.11±0.2, 10.77±0.2, 13.48±0.2, 16.8±0.2, 17.14±0.2, 17.74±0.2 and 19.69±0.2; even more preferably having 2θ angle values of 8.69±0.2, 9.01±0.2, 10.11±0.2, 10.77±0.2, 13.48±0.2, 16.8±0.2, 17.14±0.2, 17.74±0.2, 19.69±0.2, 22.09±0.2 and 23.37±0.2;
fumarate Type K, characterized by a powder X-ray diffraction pattern comprising diffraction peaks having 2θ angle values of 7.84±0.2, 14.69±0.2 and 15.75±0.2; preferably having 2θ angle values of 7.84±0.2, 8.9±0.2, 9.22±0.2, 14.69±0.2 and 15.75±0.2; more preferably having 2θ angle values of 7.84±0.2, 8.9±0.2, 9.22±0.2, 9.58±0.2, 14.69±0.2, 15.75±0.2 and 20.06±0.2; even more preferably having 2θ angle values of 7.84±0.2, 8.9±0.2, 9.22±0.2, 9.58±0.2, 14.69±0.2, 15.75±0.2, 19.65±0.2, 20.06±0.2 and 22.17±0.2; even more preferably having 2θ angle values of 7.84±0.2, 8.9±0.2, 9.22±0.2, 9.58±0.2, 14.69±0.2, 15.75±0.2, 18.7±0.2, 19.65±0.2, 20.06±0.2, 20.64±0.2 and 22.17±0.2;
fumarate Type D, characterized by a powder X-ray diffraction pattern comprising diffraction peaks having 2θ angle values of 9.64±0.2, 14.47±0.2 and 19.34±0.2; preferably having 2θ angle values of 4.83±0.2, 9.64±0.2, 13.01±0.2, 14.47±0.2 and 19.34±0.2; more preferably having 2θ angle values of 4.83±0.2, 7.92±0.2, 9.64±0.2, 13.01±0.2, 14.07±0.2, 14.47±0.2 and 19.34±0.2; even more preferably having 2θ angle values of 4.83±0.2, 7.92±0.2, 9.64±0.2, 13.01±0.2, 14.07±0.2, 14.47±0.2, 17.75±0.2, 19.34±0.2 and 20.24±0.2; even more preferably having 2θ angle values of 4.83±0.2, 7.92±0.2, 8.87±0.2, 9.64±0.2, 13.01±0.2, 14.07±0.2, 14.47±0.2, 17.75±0.2, 19.34±0.2, 20.24±0.2 and 21.88±0.2;
fumarate Type L, characterized by a powder X-ray diffraction pattern comprising diffraction peaks having 2θ angle values of 10.11±0.2, 15.16±0.2 and 20.27±0.2; preferably having 2θ angle values of 10.11±0.2, 13.98±0.2, 15.16±0.2, 20.27±0.2 and 22.69±0.2; more preferably having 2θ angle values of 10.11±0.2, 13.98±0.2, 14.14±0.2, 15.16±0.2, 18.6±0.2, 20.27±0.2 and 22.69±0.2; even more preferably having 2θ angle values of 7.89±0.2, 10.11±0.2, 13.98±0.2, 14.14±0.2, 15.16±0.2, 18.15±0.2, 18.6±0.2, 20.27±0.2 and 22.69±0.2; even more preferably having 2θ angle values of 7.89±0.2, 10.11±0.2, 13.98±0.2, 14.14±0.2, 15.16±0.2, 18.15±0.2, 18.43±0.2, 18.6±0.2, 19.86±0.2, 20.27±0.2 and 22.69±0.2;
fumarate Type F, characterized by a powder X-ray diffraction pattern comprising diffraction peaks having 2θ angle values of 8.2±0.2, 9.16±0.2 and 13.74±0.2; preferably having 2θ angle values of 8.2±0.2, 9.16±0.2, 12.06±0.2, 13.74±0.2 and 18.33±0.2; more preferably having 2θ angle values of 4.6±0.2, 8.2±0.2, 9.16±0.2, 12.06±0.2, 13.74±0.2, 18.33±0.2 and 19.97±0.2; even more preferably having 2θ angle values of 4.6±0.2, 8.2±0.2, 9.16±0.2, 12.06±0.2, 13.74±0.2, 15.33±0.2, 18.33±0.2, 19.97±0.2 and 23.33±0.2; even more preferably having 2θ angle values of 4.6±0.2, 8.2±0.2, 9.16±0.2, 12.06±0.2, 13.74±0.2, 15.33±0.2, 18.33±0.2, 19.97±0.2, 20.96±0.2, 22.06±0.2 and 23.33±0.2;
fumarate Type M, characterized by a powder X-ray diffraction pattern comprising diffraction peaks having 2θ angle values of 4.1±0.2, 6.83±0.2 and 10.23±0.2; preferably having 2θ angle values of 4.02±0.2, 4.1±0.2, 4.98±0.2, 6.83±0.2 and 10.23±0.2; more preferably having 2θ angle values of 3.21±0.2, 4.02±0.2, 4.1±0.2, 4.98±0.2, 6.52±0.2, 6.83±0.2 and 10.23±0.2; even more preferably having 2θ angle values of 3.21±0.2, 3.86±0.2, 4.02±0.2, 4.1±0.2, 4.22±0.2, 4.98±0.2, 6.52±0.2, 6.83±0.2 and 10.23±0.2; even more preferably having 2θ angle values of 3.21±0.2, 3.86±0.2, 4.02±0.2, 4.1±0.2, 4.22±0.2, 4.69±0.2, 4.98±0.2, 6.52±0.2, 6.83±0.2, 7.74±0.2 and 10.23±0.2;
fumarate Type H, characterized by a powder X-ray diffraction pattern comprising diffraction peaks having 2θ angle values of 8.13±0.2, 8.43±0.2 and 9.37±0.2; preferably having 2θ angle values of 8.13±0.2, 8.43±0.2, 9.37±0.2, 12.92±0.2 and 22.15±0.2; more preferably having 2θ angle values of 8.13±0.2, 8.43±0.2, 9.37±0.2, 11.71±0.2, 12.92±0.2, 20.13±0.2 and 22.15±0.2; even more preferably having 2θ angle values of 8.13±0.2, 8.43±0.2, 9.37±0.2, 11.71±0.2, 12.21±0.2, 12.92±0.2, 20.13±0.2, 22.15±0.2 and 23.2±0.2; even more preferably having 2θ angle values of 8.13±0.2, 8.43±0.2, 9.37±0.2, 11.71±0.2, 12.21±0.2, 12.92±0.2, 15.69±0.2, 20.13±0.2, 22.15±0.2 and 23.2±0.2;
fumarate Type J, characterized by a powder X-ray diffraction pattern comprising diffraction peaks having 2θ angle values of 4.35±0.2, 8.58±0.2 and 12.84±0.2; preferably having 2θ angle values of 4.35±0.2, 8.58±0.2, 12.84±0.2, 21.44±0.2 and 25.91±0.2; more preferably having 2θ angle values of 4.35±0.2, 7.61±0.2, 8.58±0.2, 10.08±0.2, 12.84±0.2, 21.44±0.2 and 25.91±0.2; even more preferably having 2θ angle values of 4.35±0.2, 7.61±0.2, 8.58±0.2, 10.08±0.2, 12.84±0.2, 20.26±0.2, 21.44±0.2, 22.73±0.2 and 25.91±0.2; even more preferably having 2θ angle values of 4.35±0.2, 7.61±0.2, 8.58±0.2, 10.08±0.2, 12.84±0.2, 13.33±0.2, 17.08±0.2, 20.26±0.2, 21.44±0.2, 22.73±0.2 and 25.91±0.2; and
fumarate Type E, characterized by a powder X-ray diffraction pattern comprising diffraction peaks having 2θ angle values of 8.93±0.2, 13.48±0.2 and 13.99±0.2; preferably having 2θ angle values of 8.93±0.2, 13.48±0.2, 13.99±0.2, 14.5±0.2 and 18.7±0.2; more preferably having 2θ angle values of 7.56±0.2, 8.93±0.2, 9.3±0.2, 13.48±0.2, 13.99±0.2, 14.5±0.2, 18.7±0.2 and 20.7±0.2; even more preferably having 2θ angle values of 8.93±0.2, 9.3±0.2, 13.48±0.2, 13.99±0.2, 14.5±0.2, 18.7±0.2, 19±0.2 and 20.7±0.2.

30. A crystalline form of claim 20, substantially characterized by a powder X-ray diffraction pattern selected from the group consisting of FIGS. 2, 8, 10, 12, 14, 16, 18, 19, 20, 21, 25, 26, 30, 31, 34, 35, 38, 41, 42, 43, 44 and 45.

31. A pharmaceutical composition comprising a therapeutically effective amount of crystalline form according to claim 20, and optionally one or more pharmaceutically acceptable carrier(s).

32. A method for treating or preventing a disorder or a disease selected from inflammatory disorder, autoimmune disease, or a cancer, comprising administering a subject in need thereof a therapeutically effective amount of the crystalline form according to the pharmaceutical composition of claim 31.

33. A process for the preparation of the crystalline form of claim 28, comprising:
(a) step (1) fumarate is dissolved in a mixture of EtOAc/MeOH to form a clear solution, and the clear solution is then slow evaporated to give the crystalline; or
(b) step (2) Fumarate is dissolved in EtOH to form a solution, the solution is concentrated, and the resulting material is stirred to give the crystalline; or
(c) step (3) Fumarate is dissolved in EtOH, to the mixture is added n-heptane, and the mixture is stirred to give the crystalline; or
(d) step (4) Fumarate is placed in a water vapour atmosphere to give the crystalline; or
(e) step (5) Fumarate is dissolved in a mixture of 1,4-dioxane and water, the mixture is stirred at room temperature and at −8° C.˜0° C. to give the crystalline; or
(f) step (6) Fumarate is dissolved in EtOH at 60° C.˜90° C., and a the resulting clear solution is stirred at to give the crystalline; or
(g) step (7) fumarate is dissolved in NMP, to a resulting clear solution is added EtOAc, and the resulting mixture is stirred to give the crystalline; or
(h) step (8) Fumarate is placed in a EtOH vapour atmosphere to give the crystalline.

34. The process for the preparation of the crystalline form of claim 33, wherein:
(a) the time of step (1) is about 5-about 10 days, preferably 7 days; and/or EtOAc/MeOH is 1:1 to 4:1, preferably is 2:1;
(b) step (2) further comprises rinsing the solid with EtOH and drying to give the crystalline;
(c) the temperature of step (3) is room temperature and/or the time of step (3) is overnight;
(d) the time of step (4) is 6-10 days, preferably is 8 days;
(e) the ratio of 1,4-dioxane and water of step (5) is 8:1 to 10:1, preferably is 9/1;
(f) the time of step (6) is 1-5 days, preferably is 2 days;
(g) the temperature of step (7) is room temperature and/or the time of step (3) is overnight; and/or
(h) the time of step (8) is 6-10 days, preferably is 8 days; and/or step (8) comprises air-drying at RT overnight.

35. A process for the preparation of the crystalline form of claim 28, comprising:
step (a): a crystalline form is heated to about 80 to about 160° C.; and optionally further comprising
step (b): the crystalline form is cooled to about 10 to about 40° C.

36. The process for the preparation of the crystalline form of claim 35, wherein:
(a) the crystalline form of step (a) is heated to about 100 to about 150° C.; and the crystalline form of step (b) is cooled to about 20 to about 35° C.;
(b) the crystalline form of step (a) is heated to about 140° C.; and the crystalline form of step (b) is cooled to about 30° C.;
(c) the process is under N₂ atmosphere;
(d) the starting crystalline is selected from the group consisting of type A, D, F, G, H, J, E and I; and/or
(e) the starting crystalline is selected from the group consisting of type A, D, and F.

37. (canceled)

38. (canceled)
